(19) United States
(12) Patent Application Publication
(10) Pub. No.: US 2025/0026820 A1
(43) Pub. Date: Jan. 23, 2025

(54) ANTI-TGFbeta ANTIBODY AND PREPARATION METHOD AND APPLICATION THEREOF

(71) Applicant: WUHAN YZY BIOPHARMA CO., LTD., Wuhan, Hubei (CN)

(72) Inventors: Jian SHI, Wuhan, Hubei (CN); Lijuan FANG, Wuhan, Hubei (CN); Yongxiang YAN, Wuhan, Hubei (CN); Jing ZHANG, Wuhan, Hubei (CN); Shan HUA, Wuhan, Hubei (CN); Pengfei ZHOU, Wuhan, Hubei (CN)

(21) Appl. No.: 18/684,101

(22) PCT Filed: Aug. 19, 2021
(86) PCT No.: PCT/CN2021/113494

§ 371 Date: Feb. 15, 2024

(51) Int. Cl. C07K 16/22; A61K 47/68; A61P 35/00

(52) U.S. Cl. CPC C07K 16/22; A61K 47/6845; A61P 35/00; C07K 2317/24; C07K 2317/31; C07K 2317/35; C07K 2317/522; C07K 2317/565; C07K 2317/76; C07K 2317/92

(57) ABSTRACT

The present invention relates to an anti-TGFβ antibody, such as B2G8, comprising CDR1, CDR2 and CDR3 contained in a sequence shown in SEQ ID NO: 7, preferably according to the Kabat and IMGT numbering system, comprising CDR1 as shown in SEQ ID NO: 49, CDR2 as shown in SEQ ID NO: 50 and CDR3 as shown in SEQ ID NO: 51, and an application of said anti-TGFβ antibody.

### TECHNICAL FIELD

The present invention relates to the field of pharmaceuticals, and particularly relates to an antibody against TGF-β (transforming growth factor beta), a method for preparing the same and use thereof.

### BACKGROUND

TGF-β (transforming growth factor β) is a multifunctional cytokine, and plays an important role in regulating the growth and differentiation of cells. TGF-β is a dimer composed of two structurally identical or similar subunits, each with a molecular weight of 12.5 kDa, linked by a disulfide bond. At least four subtypes, TGF-β1, TGF-β2, TGF-β3, and TGF-β1β2, are found in mammals. The genes of human TGF-β1, TGF-β2, and TGF-β3 are located at chromosomes 1993, 1q41, and 14q24, respectively. The homology of human and mouse TGF-β1 is up to 99%. TGF-β, after binding to a receptor, mediates a series of biological responses via Smad and non-Smad signaling pathways, including promoting cell epithelium-mesenchyma transformation, promoting tissue fibrosis, promoting angiogenesis, promoting the immune escape of tumors, dual effect of inhibiting cancer and promoting cancer, and the like. Since the signaling pathway mediated by overactivated TGF-β and its receptor has important pathophysiological roles in the development and progression of various diseases such as malignant tumors and tissue fibrosis, inhibitors against this molecule have been used to block this signaling pathway, thus providing a therapeutic approach to the control of these diseases.

Transforming growth factor β1 (TGF-β1) is widely involved in various pathophysiological processes in vivo, is closely related to the development and progression of various diseases such as inflammation, trauma, and organ fibrosis, and particularly has a regulating effect in the development and progression of tumors. Like TGF-β1 and TGF-β3, TGF-β2 also plays an important role in cell proliferation and differentiation, embryonic development, extracellular matrix formation, bone formation and reconstruction, tumor inhibition, metastasis, and diffusion, and the like. TGF-β2 has been identified as being able to inhibit the growth of IL-2 dependent T cells and inhibit immune surveillance during tumor development, thereby promoting tumor growth in an autocrine manner. TGF-β2 can influence the activity of killer cells and reduce the expression of cytokines such as IL-2, IL-6, IL-10, and IFN-7. TGF-β3 stimulates target cells (fibroblasts, vascular endothelial cells, and the like) to synthesize extracellular matrix and accelerate vascularization in the process of tissue repair, and promotes wound healing and relieves scar formation. TGF-β3 can promote morphogenesis in mammalian embryonic development, and has important effects on vertebration, acral sprouting, teething, facial bone formation, and heart valve formation. TGF-β3 can regulate bone formation and can influence adult bone regeneration.

Drugs targeting TGF-β have a wide range of indications, including nephropathy, systemic scleroderma, solid tumors, idiopathic pulmonary fibrosis, diabetes, and the like, but no drug targeting TGF-β is currently on the market.

### SUMMARY

The present invention provides a specific antibody against TGFβ. Specifically, the present invention provides the following aspects:

1. An anti-TGFβ antibody, comprising or consisting of a sequence selected from the group consisting of:
(1) B2G8, comprising a CDR1, a CDR2, and a CDR3 comprised in a sequence set forth in SEQ ID NO: 7, preferably comprising a CDR1 set forth in SEQ ID NO: 49, a CDR2 set forth in SEQ ID NO: 50, and a CDR3 set forth in SEQ ID NO: 51, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 49, the CDR2 set forth in SEQ ID NO: 50, and/or the CDR3 set forth in SEQ ID NO: 51 and retaining the binding affinity to TGFβ, preferably comprising the CDR1 set forth in SEQ ID NO: 49, a variant of the CDR2 set forth in SEQ ID NO: 50, and the CDR3 set forth in SEQ ID NO: 51, wherein the variant of the CDR2 set forth in SEQ ID NO: 50 is that the 13th amino acid of an amino acid sequence set forth in SEQ ID NO: 50 is replaced with alanine, or comprising a CDR1 set forth in SEQ ID NO: 49, a CDR2 set forth in SEQ ID NO: 50, and a CDR3 set forth in SEQ ID NO: 51, wherein amino acids 5-8 in the CDR1 set forth in SEQ ID NO: 49, amino acids 1-5 and amino acids 7-9 in the CDR2 set forth in SEQ ID NO: 50, and amino acid 1 and amino acids 4-9 in the CDR3 set forth in SEQ ID NO: 51 are selected from amino acid X, wherein the amino acid X is selected from the group consisting of Ala, Arg, Asn, Asp, Cys, Gln, Glu, Gly, His, Ile, Leu, Lys, Met, Phe, Pro, Ser, Thr, Trp, Tyr, and Val,
(2) B1B8, comprising a CDR1, a CDR2, and a CDR3 comprised in a sequence set forth in SEQ ID NO: 4, preferably comprising a CDR1 set forth in SEQ ID NO: 40, a CDR2 set forth in SEQ ID NO: 41, and a CDR3 set forth in SEQ ID NO: 42, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 40, the CDR2 set forth in SEQ ID NO: 41, and/or the CDR3 set forth in SEQ ID NO: 42 and retaining the binding affinity to TGFβ, or
comprising a CDR1 set forth in SEQ ID NO: 40, a CDR2 set forth in SEQ ID NO: 41, and a CDR3 set forth in SEQ ID NO: 42, wherein amino acid 3 and amino acids 5-8 in the CDR1 set forth in SEQ ID NO: 40, amino acids 1, 3, and 5-10 in the CDR2 set forth in SEQ ID NO: 41, and amino acids 4-9 and 11 in the CDR3 set forth in SEQ ID NO: 42 are selected from amino acid X, wherein the amino acid X is selected from the group consisting of Ala, Arg, Asn, Asp, Cys, Gln, Glu, Gly, His, Ile, Leu, Lys, Met, Phe, Pro, Ser, Thr, Trp, Tyr, and Val,
(3) B3F3, comprising a CDR1, a CDR2, and a CDR3 comprised in a sequence set forth in SEQ ID NO: 16, preferably comprising a CDR1 set forth in SEQ ID NO: 76, a CDR2 set forth in SEQ ID NO: 77, and a CDR3 set forth in SEQ ID NO: 78, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 76, the CDR2 set forth in SEQ ID NO: 77, and/or the CDR3 set forth in SEQ ID NO: 78 and retaining the binding affinity to TGFβ; or
comprising a CDR1 set forth in SEQ ID NO: 76, a CDR2 set forth in SEQ ID NO: 77, and a CDR3 set forth in SEQ ID NO: 78, wherein amino acids 2-3 and amino acids 5-8 in the CDR1 set forth in SEQ ID NO: 76, amino acids 2-4 and amino acid 6 in the CDR2 set forth in SEQ ID NO: 77, and amino acid 1 and amino acids 3-8 in the CDR3 set forth in SEQ ID NO: 78 are selected from amino acid X, wherein the amino acid X is selected from the group consisting of Ala, Arg, Asn, Asp, Cys, Gln, Glu, Gly, His, Ile, Leu, Lys, Met, Phe, Pro, Ser, Thr, Trp, Tyr, and Val,
(4) B2A1, comprising a CDR1, a CDR2, and a CDR3 comprised in a sequence set forth in SEQ ID NO: 1, preferably comprising a CDR1 set forth in SEQ ID NO: 31, a CDR2 set forth in SEQ ID NO: 32, and a CDR3 set forth in SEQ ID NO: 33, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 31, the CDR2 set forth in SEQ ID NO: 32, and/or the CDR3 set forth in SEQ ID NO: 33 and retaining the binding affinity to TGFβ;
(5) B1F8, comprising a CDR1, a CDR2, and a CDR3 comprised in a sequence set forth in SEQ ID NO: 5, preferably comprising a CDR1 set forth in SEQ ID NO: 43, a CDR2 set forth in SEQ ID NO: 44, and a CDR3 set forth in SEQ ID NO: 45, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 43, the CDR2 set forth in SEQ ID NO: 44, and/or the CDR3 set forth in SEQ ID NO: 45 and retaining the binding affinity to TGFβ;
(6) B3D6, comprising a CDR1, a CDR2, and a CDR3 comprised in a sequence set forth in SEQ ID NO: 6, preferably comprising a CDR1 set forth in SEQ ID NO: 46, a CDR2 set forth in SEQ ID NO: 47, and a CDR3 set forth in SEQ ID NO: 48, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 46, the CDR2 set forth in SEQ ID NO: 47, and/or the CDR3 set forth in SEQ ID NO: 48 and retaining the binding affinity to TGFβ;
(7) B2A9, comprising a CDR1, a CDR2, and a CDR3 comprised in a sequence set forth in SEQ ID NO: 2, preferably comprising a CDR1 set forth in SEQ ID NO: 34, a CDR2 set forth in SEQ ID NO: 35, and a CDR3 set forth in SEQ ID NO: 36, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 34, the CDR2 set forth in SEQ ID NO: 35, and/or the CDR3 set forth in SEQ ID NO: 36 and retaining the binding affinity to TGFβ;
(8) B2C2, comprising a CDR1, a CDR2, and a CDR3 comprised in a sequence set forth in SEQ ID NO: 8, preferably comprising a CDR1 set forth in SEQ ID NO: 52, a CDR2 set forth in SEQ ID NO: 53, and a CDR3 set forth in SEQ ID NO: 54, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 52, the CDR2 set forth in SEQ ID NO: 53, and/or the CDR3 set forth in SEQ ID NO: 54 and retaining the binding affinity to TGFβ;
(9) B1B7, comprising a CDR1, a CDR2, and a CDR3 comprised in a sequence set forth in SEQ ID NO: 9, preferably comprising a CDR1 set forth in SEQ ID NO: 55, a CDR2 set forth in SEQ ID NO: 56, and a CDR3 set forth in SEQ ID NO: 57, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 55, the CDR2 set forth in SEQ ID NO: 56, and/or the CDR3 set forth in SEQ ID NO: 57 and retaining the binding affinity to TGFβ;
(10) B1C5, comprising a CDR1, a CDR2, and a CDR3 comprised in a sequence set forth in SEQ ID NO: 10, preferably comprising a CDR1 set forth in SEQ ID NO: 58, a CDR2 set forth in SEQ ID NO: 59, and a CDR3 set forth in SEQ ID NO: 60, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 58, the CDR2 set forth in SEQ ID NO: 59, and/or the CDR3 set forth in SEQ ID NO: 60 and retaining the binding affinity to TGFβ;
(11) B2F6, comprising a CDR1, a CDR2, and a CDR3 comprised in a sequence set forth in SEQ ID NO: 11, preferably comprising a CDR1 set forth in SEQ ID NO: 61, a CDR2 set forth in SEQ ID NO: 62, and a CDR3 set forth in SEQ ID NO: 63, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 61, the CDR2 set forth in SEQ ID NO: 62, and/or the CDR3 set forth in SEQ ID NO: 63 and retaining the binding affinity to TGFβ;
(12) B2H1, comprising a CDR1, a CDR2, and a CDR3 comprised in a sequence set forth in SEQ ID NO: 12, preferably comprising a CDR1 set forth in SEQ ID NO: 64, a CDR2 set forth in SEQ ID NO: 65, and a CDR3 set forth in SEQ ID NO: 66, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 64, the CDR2 set forth in SEQ ID NO: 65, and/or the CDR3 set forth in SEQ ID NO: 66 and retaining the binding affinity to TGFβ;
(13) B2A2, comprising a CDR1, a CDR2, and a CDR3 comprised in a sequence set forth in SEQ ID NO: 13, preferably comprising a CDR1 set forth in SEQ ID NO: 67, a CDR2 set forth in SEQ ID NO: 68, and a CDR3 set forth in SEQ ID NO: 69, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 67, the CDR2 set forth in SEQ ID NO: 68, and/or the CDR3 set forth in SEQ ID NO: 69 and retaining the binding affinity to TGFβ;
(14) B3D2, comprising a CDR1, a CDR2, and a CDR3 comprised in a sequence set forth in SEQ ID NO: 14, preferably comprising a CDR1 set forth in SEQ ID NO: 70, a CDR2 set forth in SEQ ID NO: 71, and a CDR3 set forth in SEQ ID NO: 72, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 70, the CDR2 set forth in SEQ ID NO: 71, and/or the CDR3 set forth in SEQ ID NO: 72 and retaining the binding affinity to TGFβ;
(15) B3C5, comprising a CDR1, a CDR2, and a CDR3 comprised in a sequence set forth in SEQ ID NO: 15, preferably comprising a CDR1 set forth in SEQ ID NO: 73, a CDR2 set forth in SEQ ID NO: 74, and a CDR3 set forth in SEQ ID NO: 75, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 73, the CDR2 set forth in SEQ ID NO: 74, and/or the CDR3 set forth in SEQ ID NO: 75 and retaining the binding affinity to TGFβ;
(16) B2C9, comprising a CDR1, a CDR2, and a CDR3 comprised in a sequence set forth in SEQ ID NO: 3, preferably comprising a CDR1 set forth in SEQ ID NO: 37, a CDR2 set forth in SEQ ID NO: 38, and a CDR3 set forth in SEQ ID NO: 39, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 37, the CDR2 set forth in SEQ ID NO: 38, and/or the CDR3 set forth in SEQ ID NO: 39 and retaining the binding affinity to TGFβ, and
(17) B3F1, comprising a CDR1, a CDR2, and a CDR3 comprised in a sequence set forth in SEQ ID NO: 17, preferably comprising a CDR1 set forth in SEQ ID NO: 79, a CDR2 set forth in SEQ ID NO: 80, and a CDR3 set forth in SEQ ID NO: 81, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 79, the CDR2 set forth in SEQ ID NO: 80, and/or the CDR3 set forth in SEQ ID NO: 81 and retaining the binding affinity to TGFβ.

2. The anti-TGFβ antibody according to item 1, comprising a sequence selected from SEQ ID NOs: 1-17 or a variant thereof, or consisting of a sequence selected from SEQ ID NOs: 1-17 or a variant thereof, wherein the variant is a sequence having at least 80%, 85%, 90%, 91%, 92%, 93%, 94%, 95%, 96%, 97%, 98%, or 99% homology to the amino acid sequence set forth in the SEQ ID NOs and retaining the binding affinity to TGFβ, or an amino acid sequence having one or more (preferably 1, 2, 3, 4, 5, 6, 7, 8, 9 or 10) conservative amino acid mutations (preferably substitutions, insertions or deletions) as compared to the amino acid sequence set forth in the SEQ ID NOs and retaining the binding affinity to TGFβ.

3. A humanized anti-TGFβ antibody, comprising the CDR1, the CDR2, and the CDR3 of the anti-TGFβ antibody according to item 1, preferably comprising an FR region from Germline IGHV3-23*01 of human IgG, preferably further comprising an Fc constant region, wherein the constant region comprises a sequence set forth in SEQ ID NO: 18 or the human IgG1 heavy chain Fc constant region (GenBank No. AK303185.1), wherein more preferably, the humanized anti-TGFβ antibody comprises or consists of a humanized sequence set forth in SEQ ID NO: 20; further preferably, serine Ser at position 13 of a CDR2 of the humanized sequence set forth in SEQ ID NO: 20 is mutated to alanine Ala; most preferably, the humanized anti-TGFβ antibody comprises or consists of a humanized sequence selected from SEQ ID NOs: 21-23.

4. A bivalent or multivalent anti-TGFβ antibody, comprising 2 or more of the anti-TGFβ antibody according to any one of items 1-2, or comprising 2 or more of the humanized anti-TGFβ antibody according to item 3, wherein preferably, the bivalent anti-TGFβ antibody has a sequence selected from the group consisting of: (1) the sequences set forth in SEQ ID NOs: 22, 19, 18, 19, and 22 connected end-to-end in order from the N-terminus to the C-terminus; (2) the sequences set forth in SEQ ID NOs: 4, 19, 18, 19, and 4 connected end-to-end in order from the N-terminus to the C-terminus; (3) the sequences set forth in SEQ ID NOs: 23, 19, 18, 19, and 23 connected end-to-end in order from the N-terminus to the C-terminus; (4) the sequences set forth in SEQ ID NOs: 21, 19, 18, 19, and 21 connected end-to-end in order from the N-terminus to the C-terminus; (5) the sequences set forth in SEQ ID NOs: 7, 19, 18, 19, and 7 connected end-to-end in order from the N-terminus to the C-terminus; and (6) the sequences set forth in SEQ ID NOs: 16, 19, 18, 19, and 16 connected end-to-end in order from the N-terminus to the C-terminus.

5. A multispecific antibody, comprising the anti-TGFβ antibody according to any one of items 1-2 or the humanized anti-TGFβ antibody according to item 3, preferably a bispecific antibody comprising a first antibody against a first antigen TGFβ and a second antibody against a second antigen, wherein the first antibody is selected from the anti-TGFβ antibody according to any one of items 1-2 or the humanized anti-TGFβ antibody according to item 3, and the second antigen is selected from an immune cell surface antigen, a tumor antigen, a virus, a bacterium, an endotoxin, a cytokine, or a combination thereof, more preferably selected from: PD-L1, PD-1, VEGFA, IL-10, IL-10R, BCMA, VEGF, TGF-β, CTLA-4, LAG-3, TIGIT, CEA, CD38, SLAMF7, B7-H3, Her2, EpCAM, CD19, CD20, CD30, CD33, CD47, CD52, CD133, EGFR, GD2, GD3, GM2, RANKL, CD3, and/or CD16a, wherein preferably, the second antigen is VEGF; more preferably, the second antibody is selected from an anti-VEGF antibody comprising (i) an HCDR1 set forth in SEQ ID NO: 82, an HCDR2 set forth in SEQ ID NO: 83, an HCDR3 set forth in SEQ ID NO: 84, an LCDR1 set forth in SEQ ID NO: 85, an LCDR2 set forth in SEQ ID NO: 86, and an LCDR3 set forth in SEQ ID NO: 87, or (ii) an HCDR1 set forth in SEQ ID NO: 88, an HCDR2 set forth in SEQ ID NO: 89, an HCDR3 set forth in SEQ ID NO: 90, an LCDR1 set forth in SEQ ID NO: 91, an LCDR2 set forth in SEQ ID NO: 92, and an LCDR3 set forth in SEQ ID NO: 93; more preferably, the anti-VEGF antibody comprises (a) a heavy chain variable region set forth in SEQ ID NO: 24 or a sequence having at least 80%, 85%, 90%, 91%, 92%, 93%, 94%, 95%, 96%, 97%, 98%, or 99% homology to the heavy chain variable region set forth in SEQ ID NO: 24, and a light chain variable region set forth in SEQ ID NO: 27 or a sequence having at least 80%, 85%, 90%, 91%, 92%, 93%, 94%, 95%, 96%, 97%, 98%, or 99% homology to the light chain variable region set forth in SEQ ID NO: 27, or (b) a heavy chain variable region set forth in SEQ ID NO: 29 or a sequence having at least 80%, 85%, 90%, 91%, 92%, 93%, 94%, 95%, 96%, 97%, 98%, or 99% homology to the heavy chain variable region set forth in SEQ ID NO: 29, and a light chain variable region set forth in SEQ ID NO: 30 or a sequence having at least 80%, 85%, 90%, 91%, 92%, 93%, 94%, 95%, 96%, 97%, 98%, or 99% homology to the light chain variable region set forth in SEQ ID NO: 30; most preferably, the anti-VEGF antibody further comprises a CH1 set forth in SEQ ID NO: 25, a hinge region set forth in SEQ ID NO: 26, an Fc constant region set forth in SEQ ID NO: 18, and a light chain constant region set forth in SEQ ID NO: 28.

6. A polynucleotide, encoding the anti-TGFβ antibody according to any one of items 1-2 or the humanized anti-TGFβ antibody according to item 3, or the bivalent or multivalent anti-TGFβ antibody according to item 4, or the multispecific antibody according to item 5.

7. A vector, comprising the polynucleotide according to item 6.

8. A host cell, comprising the vector according to item 7.

9. A conjugate, comprising the anti-TGFβ antibody according to any one of items 1-2, the humanized anti-TGFβ antibody according to item 3, the bivalent or multivalent anti-TGFβ antibody according to item 4, or the multispecific antibody according to item 5, and a conjugated moiety, wherein the conjugated moiety is a purification tag (e.g., His tag), a detectable label, a drug, a toxin, a cytokine, an enzyme or a combination thereof, preferably, the conjugated moiety is a radioisotope, a fluorescent substance, a chemiluminescent substance, a colored substance, a chemotherapeutic agent, a biotoxin, polyethylene glycol or an enzyme.

10. A kit, comprising the anti-TGFβ antibody according to any one of items 1-2, the humanized anti-TGFβ antibody according to item 3, the bivalent or multivalent anti-TGFβ antibody according to item 4, the multispecific antibody according to item 5, or the conjugate according to item 9, wherein preferably, the kit further comprises a second antibody specifically recognizing the anti-TGFβ antibody according to any one of items 1-2, the humanized anti-TGFβ antibody according to item 3, the bivalent or multivalent anti-TGFβ antibody according to item 4, the multispecific antibody according to item 5, or the conjugate according to item 9; optionally, the second antibody further comprises a detectable label, such as a radioisotope, a fluorescent substance, a chemiluminescent substance, a colored substance or an enzyme; preferably, the kit is configured for detecting the presence or level of VEGF in a sample; or the kit comprises (1) the anti-TGFβ antibody according to any one of items 1-2, the humanized anti-TGFβ antibody according to item 3, the bivalent or multivalent anti-TGFβ antibody according to item 4, the multispecific antibody according to item 5, or the conjugate according to item 9, and (2) an antibody or an antigen-binding fragment thereof against another antigen, and/or a cytotoxic agent, and/or a chemotherapeutic agent, and optionally, a package insert.

11. A pharmaceutical composition, comprising the anti-TGFβ antibody according to any one of items 1-2, the humanized anti-TGFβ antibody according to item 3, the bivalent or multivalent anti-TGFβ antibody according to item 4, the multispecific antibody according to item 5, or the conjugate according to item 9, wherein optionally, the pharmaceutical composition further comprises a pharmaceutically acceptable carrier and/or excipient; preferably, the pharmaceutical composition is in a form suitable for administration through subcutaneous injection, intradermal injection, intravenous injection, intramuscular injection or intralesional injection.

12. Use of the anti-TGFβ antibody according to any one of items 1-2, the humanized anti-TGFβ antibody according to item 3, the bivalent or multivalent anti-TGFβ antibody according to item 4, the multispecific antibody according to item 5, or the conjugate according to item 9 in a medicament for treating and/or preventing a tumor (e.g., treating a solid tumor), diabetes, systemic scleroderma, nephropathy, idiopathic pulmonary fibrosis, and/or multiple fibrosis.

It is to be understood that within the scope of the present invention, the above technical features of the present invention and the technical features specifically described hereinafter (as in the examples) may be combined with each other to constitute a new or preferred technical scheme. Due to the limited space, such schemes are not described herein.

The terms referred to in the present invention have the conventional meanings understood by those skilled in the art. When a term has two or more definitions as used and/or acceptable in the art, the definitions of the terms used herein are intended to include all meanings.

It will be understood by those of ordinary skills in the art that the CDR regions of an antibody are responsible for the binding specificity of the antibody for an antigen. For a given heavy or light chain variable region sequence of an antibody, there are several methods for determining the CDR regions of the antibody, including the Kabat, IMGT, Chothia and AbM numbering systems. However, the application of all the definitions of CDRs for an antibody or variants thereof shall fall within the scope of the terms defined and used herein. If the amino acid sequence of the variable region of the antibody is given, those skilled in the art can generally determine a particular CDR, without relying on any experimental data beyond the sequence itself.

The antibody of the present invention may be (i) a polypeptide in which one or more conservative or non-conservative amino acid residues (preferably conservative amino acid residues) are substituted, or (ii) a polypeptide having a substituted group in one or more amino acid residues, or (iii) a polypeptide formed by fusing a mature polypeptide with another compound (such as a compound that prolongs the half-life of the polypeptide, e.g., polyethylene glycol), or (iv) a polypeptide formed by fusing an additional amino acid sequence with the polypeptide sequence (such as a leader sequence, a secretion sequence, a sequence for purifying the polypeptide, a proteinogenic sequence or a fusion protein formed with 6His tag). Such fragments, derivatives and analogs are well known to those skilled in the art according to the teachings of the present invention.

A “conservative amino acid substitution” is one in which an amino acid residue is substituted with an amino acid residue having a similar side chain. Families of amino acid residues having similar side chains have been defined in the art, including basic side chains (e.g., lysine, arginine and histidine), acidic side chains (e.g., aspartic acid and glutamic acid), uncharged polar side chains (e.g., glycine, asparagine, glutamine, serine, threonine, tyrosine and cysteine), non-polar side chains (e.g., alanine, valine, leucine, isoleucine, proline, phenylalanine, methionine and tryptophan), branched p side chains (e.g., threonine, valine and isoleucine) and aromatic side chains (e.g., tyrosine, phenylalanine, tryptophan and histidine). Thus, non-essential amino acid residues of an immunoglobulin polypeptide are preferably substituted with other amino acid residues from the same side chain family. In other embodiments, a string of amino acids may be substituted with a structurally similar string of amino acids that differ in sequence and/or composition of the side chain family.

Non-limiting examples of conservative amino acid substitutions are provided in the following table, where a similarity score of 0 or higher indicates that there is a conservative substitution between the two amino acids.

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

In some embodiments, the conservative substitution is preferably a substitution in which one amino acid within the following groups (a)-(e) is substituted with another amino acid residue within the same group: (a) small aliphatic, non-polar or weakly polar residues: Ala, Ser, Thr, Pro and Gly, (b) polar, negatively charged residues and (uncharged) amides thereof: Asp, Asn, Glu and Gln, (c) polar, positively charged residues: His, Arg and Lys, (d) bulky aliphatic, nonpolar residues: Met, Leu, Ile, Val and Cys, and (e) aromatic residues: Phe, Tyr and Trp.

Particularly preferred conservative substitutions are as follows: Ala is substituted with Gly or Ser; Arg is substituted with Lys; Asn is substituted with Gln or His; Asp is substituted with Glu; Cys is substituted with Ser; Gln is substituted with Asn; Glu is substituted with Asp; Gly is substituted with Ala or Pro; His is substituted with Asn or Gln; Ile is substituted with Leu or Val; Leu is substituted with Ile or Val; Lys is substituted with Arg, Gln or Glu; Met is substituted with Leu, Tyr or Ile; Phe is substituted with Met, Leu or Tyr; Ser is substituted with Thr; Thr is substituted with Ser; Trp is substituted with Tyr; Tyr is substituted with Trp; and/or Phe is substituted with Val, Ile or Leu.

In some embodiments, the antibody of the present invention may be conjugated with a therapeutic agent (e.g., a chemotherapeutic agent such as cisplatin and carboplatin), a prodrug, a peptide, a protein, an enzyme, a virus, a lipid, a biological response modifier, a pharmaceutical agent or PEG. The antibody of the present invention can be linked or fused to a therapeutic agent, and the therapeutic agent may comprise a detectable label such as a radioactive label, an immunomodulator, a hormone, an enzyme, an oligonucleotide, a photoactive therapeutic or diagnostic agent, a cytotoxic agent that can be a drug or a toxin, an ultrasound enhancing agent, a nonradioactive label, a combination thereof and other such ingredients known in the art.

Compared with the prior art, the present invention has the following beneficial effects.

The anti-TGFβ antibody of the present invention has excellent biological activity and specificity and still retains high affinity for TGFβ. The expression level is significantly improved. The antibody has good stability, particularly in acidic and heating conditions.

### BRIEF DESCRIPTION OF THE DRAWINGS

FIG. 1. Inhibition of the biological activity of TGF-β1 by the antibody.

FIG. 2. Inhibition of the biological activity of TGF-β2 by the antibody.

FIG. 3. Inhibition of the biological activity of TGF-β3 by the antibody.

FIG. 4. Inhibition of the biological activity of TGF-β1 by the humanized single-domain antibody.

FIG. 5. In vivo efficacy of the bispecific antibody G6-Fc-huG892 against mouse subcutaneous tumors CT-26 and EMT-6.

FIG. 6. In vivo efficacy of the bispecific antibody Beva-Fc-huG892 against mouse xenograft tumor A549.

FIG. 7. Inhibition of the biological activity of VEGFA-induced HUVEC cell proliferation by the bispecific antibody.

FIG. 8. Inhibition of the biological activity of TGFβ 1/2/3 by the bispecific antibody.

### DETAILED DESCRIPTION

The present invention will be described in detail below by way of examples. It will be understood by those of ordinary skills in the art that the following examples are for illustrative purposes only. The spirit and scope of the present invention are defined by the claims. Unless otherwise stated, the methods used in the following examples are conventional methods and the reagents used are commercially available reagents.

### Example 1: Screening for Heavy Chain Single-Domain Antibody Against TGFβ

### 1.1 Construction of Phage Library of Heavy Chain Single-Domain Antibodies:

TGFβ1 (Z03411), TGFβ2 (Z03429), and TGFβ3 (Z03430) proteins used for immunization were purchased from GenScript. Two Vicugna pacos (alpaca) were chosen for antigen immunization. The first immunization was performed with 1 mg of TGFβ3, and the next three immunizations were with an equal ratio mixture of TGFβ1, TGFβ2, and TGFβ3 (0.5 mg each). After 4 immunizations, lymphocytes from 100 mL peripheral blood of the Vicugna pacos were extracted, total RNA was extracted with an RNAiso Plus reagent (TAKARA, 9109), and the extracted RNA was reversely transcribed into cDNA with a PrimeScript II kit (TAKARA, 6210A). The nucleic acid fragments of the variable region and the constant region CH2 of the heavy-chain antibody with the length of 750 bp were firstly amplified using a PCR amplification method, and then the target fragments were amplified using the nucleic acid fragment with the length of 750 bp recovered in the previous step as a template, namely the variable region fragment of the heavy-chain antibody. The vector pComb3XSS (purchased from NBbiolab, China) and the target fragment were separately subjected to enzyme digestion by SfiI. Enzyme digestion was carried out overnight at 50° C. The target fragment was recovered. The ligation was performed according to the ligation molar ratio of vector:fragment=1:3. The ligation product was electrotransformed into E. coli competent cells TG1, and 10 electrotransformation were performed on each Vicugna pacos ligation product. The library diversity was calculated by gradient dilution plating, and the phage library diversities of the two Vicugna pacos were 1.21×10⁹ and 1×10⁹, respectively. 96 clones were randomly picked from the titer plate for identification, and the result shows that the insertion rate was 100%.

### 1.2 Panning of the Heavy Chain Single-Domain Antibody Against TGFβ 1:

The plate was coated with TGFβ1 protein at 10 μg/well at 4° C. overnight. The next day after being blocked with 1% BSA at room temperature for 2 h, the plate was added with 100 μL of phages (2×10⁹ pfu/well, from the constructed heavy chain single-domain antibody phage library in 1.1) and incubated at 37° C. for 1 h. Non-binding phages were washed off 5 times with PBST (0.05% Tween 20 in PBS). Finally, the phages specifically binding to TGFβ were eluted with glycine-hydrochloric acid (200 mM) and infected with E. coli TG1 in the logarithmic growth phase, and phages were generated and purified for the next round of screening. After the same screening process was repeated for 3 rounds, the obtained phages were infected with E. coli TG1 and plated. Monoclones were picked from the plate and sequenced. The protein sequence of each clone was analyzed according to the sequence alignment results. The clones with different sequences of CDR1, CDR2 and CDR3 were determined as different antibody strains, and finally a total of 17 strains of different antibodies were obtained.

TABLE 1
Amino acid sequence information of variable region of heavy
chain single-domain antibody (numbering scheme IMGT and Kabat)

| | Amino acid sequence of variable region of antibody | SEQ ID |
|---|---|---|
| Clone | (the underlined parts represent CDR regions) | NO: |
| B2A1 | QVQLVESGGGLVQPGGSLRLSCAASGITFSHYDMAWERQAPEKQRELVALITSGGK | 1 |
| | TAYADSVKGRFAISRDNAKNTVYLQMNSLKAGDTAVYYCNAHWVLTSQYWGQGTQV | |
| | TVSS | |
| | CDR1: GITFSHYDMA SEQ ID NO: 31 | |
| | CDR2: ITSGGKTAYADSVKG SEQ ID NO:32 | |
| | CDR3: NAHWVLTSQY SEQ ID NO: 33 | |
| B2A9 | QLQLVESGGGLVQPGGSLRLSCTASGSTFEIYPIYAMAWFRQTPGKQRELVALTTD | 2 |
| | VKTNYADSVKGRFTISRDNAKNTVYLQMNMLQPEDTAVYVCNVGVVRGGLQNYWGQ | |
| | GTQVTVSS | |
| | CDR1: GSTFEIYPIYAMA SEQ ID NO: 34 | |
| | CDR2: LTTDVKTNYADSVKG | |
| | CDR3: NVGVVRGGLQNY | |
| | SEQ ID NO: 36 | |
| B2C9 | EVQVVESGGGLVQPGGSLRLSCAASASVFSWSVMGWFRQAPGKQRELVAISSGDRE | 3 |
| | TYADSVKGRFTISRDNAKNTVYLQMDTLKPEDTAVYFCNAYVPVGESRGDYWGQGT | |
| | QVTVSS | |
| | CDR1: ASVFSWSVMG SEQ ID NO: 37 | |
| | CDR2: ISSGDRETYADSVKG SEQ ID NO: 38 | |
| | CDR3: NAYVPVGESRGDY SEQ ID NO:39 | |
| B1B8 | EVQVVESGGGLVQPGGSLTLSCVASGFAFSFTPMRWVRQAPGKGLEWVSSISIRGD | 4 |
| | TTDYADSVKGRFTISRDNAKNTLYLQMNSLKPEDTAVYYCSRGSSISTSTERTQGT | |
| | QVTVSS | |
| | CDR1: GFAFSFTPMR SEQ ID NO:40 | |
| | CDR2: SISIRGDTTDYADSVKG SEQ ID NO: 41 | |
| | CDR3: SRGSSISTSTE SEQ ID NO: 42 | |
| BIF8 | QVQLVESGGGLVQPGGSLRLSCAASGSIFGVFYMGWFRQAPGKQREMVAISTDDRT | 5 |
| | SYVDSVKGRFTISRDNAKNTLALQMNSLKPEDTAVYFCNLNQLGTDYWGPGTQVTV | |
| | SS | |
| | CDR1: GSIFGVFYMG SEQ ID NO: 43 | |
| | CDR2: ISTDDRTSYVDSVKG SEQ ID NO: 44 | |
| | CDR3: NLNQLGTDY SEQ ID NO: 45 | |
| B3D6 | EVQLVESGGGLVQAGGSLRLSCAASGRSFSSLAVGWFRQPPGKKREIVAAVNWSGT | 6 |
| | STYYPDSVKGRFSISRDNTKNTVYLQMNSLKPEDTAVYYCAADPDLRWLKLWGYEY | |
| | WGQGTQVTVSS | |
| | CDR1: GRSFSSLAVG SEQ ID NO: 46 | |
| | CDR2: AVNWSGTSTYYPDSVKG SEQ ID NO: 47 | |
| | CDR3: AADPDLRWLKLWGYEY SEQ ID NO: 48 | |
| B2G8 | QVQLVESGGGLVQPGGSLRLSCAASGISFSYYDMGWFRQAPGKQRELVAIVTSGGR | 7 |
| | TNYADSVKGRFTISRETAKSTMYLQMNSLKSEDTGVYYCNAHWIVTTDYWGQGTQV | |
| | TVAS | |
| | CDR1: GISFSYYDMG SEQ ID NO: 49 | |
| | CDR2: IVTSGGRTNYADSVKG SEQ ID NO: 50 | |
| | CDR3: NAHWIVTTDY SEQ ID NO: 51 | |
| B2C2 | QLQLVESGGGLVQPGGSLRLSCAGSGIAFSRVTMAWSRQAPGKQRELVALITDGNR | 8 |
| | SAYVDSVKGRFTISRDNTKKTMSLQMNDLKPEDTAVYYCVATDWVPTRRDYWGQGT | |
| | QVTVSS | |
| | CDR1: GIAFSRVTMA SEQ ID NO: 52 | |
| | CDR2: ITDGNRSAYVDSVKG SEQ ID NO: 53 | |
| | CDR3: VATDWVPTRRDY SEQ ID NO: 54 | |
| B1B7 | QVQLVESGGGLVQPGGSLTLSCTASGSMFSFYTMGWYRQATGKQRELVAGISLGGE | 9 |
| | TTYAPSVKDRFTISRDNAKNTVYLQMNSLKPEDTALYNCNAYWGEDYWGQGTQVTV | |
| | SS | |
| | CDR1: GSMFSFYT SEQ ID NO: 55 | |
| | CDR2: ISLGGETTYAPSVKD SEQ ID NO: 56 | |
| | CDR3: NAYWGEDY SEQ ID NO: 57 | |
| B1C5 | EVQLVESGGGEVRPGGSLRLSCVVSGFTKDYGAIGWFRQAPGQEREGLGCIARKDG | 10 |
| | DTWSADSVKGRFTISRDNVRNTVYLQMNALKPEDRGVYYCAGDPSALYGCSDRSSE | |
| | YQYWGQGTQVTVSS | |
| | CDR1: GFTKDYGAIG SEQ ID NO: 58 | |
| | CDR2: IARKDGDTWSADSVKG SEQ ID NO: 59 | |
| | CDR3: AGDPSALYGCSDRSSEYQY SEQ ID NO: 60 | |
| B2F6 | EVQLVESGGGLVQPGGSLRLSCAASGFTFSYYVASWYRQAPGKERELVATTFPPDG | 11 |
| | SPHYADSVKGRFAISRDNAKNTVFLQMDSLKSEDTAVYYCNARRVEGHGIYWGQGT | |
| | QVTVSS | |
| | CDR1: GFTFSYYVAS SEQ ID NO: 61 | |
| | CDR2: TTFPPDGSPHYADSVKG SEQ ID NO: 62 | |
| | CDR3: NARRVEGHGIY SEQ ID NO: 63 | |
| B2H1 | QLQLVESGGGLVQPGESLRLSCAASGIIFSASTMAWFRQAPGKQRELVALITDGDR | 12 |
| | TNYADSVKGRFTISRDNAENTVYLQMTELKFEDTAVYYCNAFWLKAYWGQGTQVTV | |
| | SS | |
| | CDR1: GIIFSASTMA SEQ ID NO: 64 | |
| | CDR2: LITDGDRTNYADSVKG SEQ ID NO: 65 | |
| | CDR3: NAFWLKAY SEQ ID NO: 66 | |
| B2A2 | EVQVVESGGAAVQPGESLRLSCAASGSIFNTATMGWFRQAPGKQREMVAIITDGGR | 13 |
| | TNYADSVKGRFTISRDATKNTVYLQMNSLNPGDTAVYYCNALTIVPRSSIYWGQGT | |
| | QVTVSS | |
| | CDR1: GSIENTATMG SEQ ID NO: 67 | |
| | CDR2: IITDGGRTNYADSVKG SEQ ID NO: 68 | |
| | CDR3: NALTIVPRSSIY SEQ ID NO: 69 | |
| B3D2 | QLQLVESGGGLVQPGGSLTLSCLASGFTFSHYPMSWVRQAPGKGLEWVSVIGSDGA | 14 |
| | IKHYSDSVKGRFAISRDNAKNTLYLQMNSLKPEDTAVYYCAEGDSRYVRNRARGQG | |
| | TQVTVSS | |
| | CDR1: GFTFSHYPMS SEQ ID NO: 70 | |
| | CDR2: IGSDGAIKHYSDSVKG SEQ ID NO: 71 | |
| | CDR3: AEGDSRYVRNRA SEQ ID NO: 72 | |
| B3C5 | QLQLVESGGGLVQPGGSLTLSCAASGFTFSHYPMSWVRQAPGKGLEWVSVIGSDGG | 15 |
| | TKIYSDSVKGRFAIARDNAKNTLYLQMNSLKPEDTAVYYCAEGDSRYVRGRTRGQG | |
| | TQVTVSS | |
| | CDR1: GFTFSHYPMS SEQ ID NO: 73 | |
| | CDR2: IGSDGGTKIYSDSVKG SEQ ID NO: 74 | |
| | CDR3: AEGDSRYVRGRT SEQ ID NO: 75 | |
| B3F3 | QVQLVESGGGLVQPGESLRLSCTASGSIFGVFYMGWFRQAPGKQRDMVAISTDDRT | 16 |
| | SYADSVKGRFTISRDNAKNTLALQMNSLKPEDTAVYFCNLNQLGTDYWGPGTQVTV | |
| | SS | |
| | CDR1: GSIFGVFYMG SEQ ID NO: 76 | |
| | CDR2: ISTDDRTSYADSVKG SEQ ID NO: 77 | |
| | CDR3: NLNQLGTDY SEQ ID NO: 78 | |
| B3F1 | QLQLVESGGGLVQPGGSLTLSCAASGFTFSLYPMSWVRQAPGKGLEWVSVIGSDGG | 17 |
| | TKHYSDSVKGRFAISRDNAKNTLYLQMNSLKPEDTAVYYCAEGDSRYVRGRTRGQG | |
| | TQVTVSS | |
| | CDR1: GFTFSLYPMS SEQ ID NO: 79 | |
| | CDR2: IGSDGGTKHYSDSVKG SEQ ID NO: 80 | |
| | CDR3: AEGDSRYVRGRT SEQ ID NO: 81 | |

### Example 2: Preliminary Evaluation and Identification of Heavy Chain Single-Domain Antibody Against TGFβ

2.1 Expression of Heavy Chain Single-Domain Antibody in Host Bacteria E. coli:

A single colony of the TG1 host bacteria of the obtained 17 strains of heavy chain single-domain antibodies was selected, inoculated, and cultured overnight. The next day the overnight strain was transferred, amplified, induced with 0.5 mM IPTG, and cultured overnight at 37° C. on a shaker. The next day, the supernatant was collected by centrifugation for assay.

### 2.2 ELISA Assay for Avidities of 17 Heavy Chain Single-Domain Antibodies:

A microplate was coated with 2 μL/well of the TGFβ1 protein solution and BSA, respectively, at 4° C. overnight; the supernatant was discarded, 300 μL of a blocking solution (PBS containing 3% BSA) was added to each well, and the plate was blocked at 37° C. for 2 h; the supernatant collected by centrifugation was added to the well of the microplate at 200 μL per well, the plate was left to stand and incubated at room temperature for 2 h, and the supernatant was discarded; the plate was washed for 3 times with 200 μL/well PBST (PBS containing 0.1% Tween 20); a diluted HRP-labeled anti-VHH secondary antibody (Genscript, A01861) was added, the secondary antibody was used after a 1:10000 dilution, the diluent was PBS containing 1% BSA, the volume was 100 μL/well, and the plate was incubated at room temperature for 1 h; the plate was washed for 5 times with 200 μL/well PBST; a TMB chromogenic solution (BD, 55214) was added at 100 μL/well for color development at 37° C. for 8 min. 2 M HCl stop solution was added at 100 μL/well. After the addition of the stop solution, the 450 nm values were read using a microplate reader within 30 min. The results are shown in Table 2 below.

TABLE 2
OD value of binding of heavy chain single-
domain antibodies to TGFβ1 antigen

| Clone | OD value of binding to TGFβ1 | OD value of binding to BSA |
|---|---|---|
| B2A1 | 1.291 | 0.057 |
| B2A9 | 1.921 | 0.061 |
| B2C9 | 1.875 | 0.055 |
| B1B8 | 1.921 | 0.063 |
| B1F8 | 1.032 | 0.07 |
| B3D6 | 1.652 | 0.067 |
| B2G8 | 1.854 | 0.064 |
| B2C2 | 2.051 | 0.058 |
| B1B7 | 1.953 | 0.067 |
| B1C5 | 2.081 | 0.052 |
| B2F6 | 1.092 | 0.053 |
| B2H1 | 2.062 | 0.057 |
| B2A2 | 1.693 | 0.064 |
| B3D2 | 1.991 | 0.061 |
| B3C5 | 1.032 | 0.051 |
| B3F3 | 2.291 | 0.071 |
| B3F1 | 1.902 | 0.051 |

### Example 3: Plasmid Preparation, Expression and Purification of Antibodies

Heavy chain antibodies were constructed by ligating the encoding DNA sequence corresponding to the heavy chain variable region (SEQ ID NOs: 1-17) of the monoclonal antibodies cloned by PCR with the encoding DNA corresponding to the human IgG1 heavy chain constant region (GenBank No. AK303185.1) to obtain an anti-TGFβ single domain heavy-chain antibody expression plasmid, typically using pcDNA3.1(−) (purchased from Invitrogen) or other eukaryotic expression vectors as the vectors. The variable region and the constant region of the heavy-chain antibody protein sequence were connected using a linker peptide, wherein the linker peptide was (GGGGS)n, n=1, 2 or 3.

Plasmid extraction was performed using EndoFree Plasmid Giga Kit (Qiagen, Cat. No. 12391), following the manufacturer's manual. CHO-S cells were cultured in CD CHO medium (Gibco, Cat. No. 10743-029) in a 37° C., 5% CO₂ incubator according to the manufacturer's manual. After the cells were prepared, CHO-S cells were co-transfected with plasmids containing heavy chain sequences to express an anti-TGFβ antibody. The day after transfection, the culture temperature was adjusted to 32° C. and 3.5% 2×EFC+(Gibco, Cat. No. A2503105) was added daily. After 14 days of culture, expression supernatants were harvested by centrifugation at 800×g. The supernatants were filtered through a 0.22 μm filter membrane, and purified by protein A affinity chromatography and cation exchange chromatography to give the anti-TGFβ antibodies in the culture supernatants. The concentration of the purified antibody was determined by UV absorbance at 280 nm and the corresponding extinction coefficient for each protein. The purity and homogeneity of the antibodies were assessed by SDS-PAGE and SE-HPLC. The antibodies were further purified by a second purification through ion exchange and SEC with Superdex 200 to prepare antibody samples of higher purities for later use.

### Example 4: Avidity of Antibodies to Bind to TGF-β

In this example, the avidities of the 17 heavy chain single-domain antibodies were assessed by ELISA.

A microplate was coated with 2 μL/well of the human TGF-β1/2/3 protein solution, respectively, at 4° C. overnight; the supernatant was discarded, 300 μL of a blocking solution (PBS containing 3% BSA) was added to each well, and the plate was blocked at 37° C. for 2 h; the antibodies were gradiently diluted, and the diluent was PBS containing 1% BSA. For example, the initial concentration of the antibody dilution was 500 nM, and diluted 10-fold to obtain 8 concentration gradients. The diluted antibody was added to the well of the microplate at 200 μL per well, the plate was left to stand and incubated at room temperature for 2 h, and the supernatant was discarded; the plate was washed for 3 times with 200 μL/well PBST (PBS containing 0.1% Tween 20); a diluted HRP-labeled anti-human Fc secondary antibody (SIGMA, A8667) was added, the secondary antibody was used after a 1:20000 dilution, the diluent was PBS containing 1% BSA, the volume was 100 μL/well, and the plate was incubated at room temperature for 1 h; the plate was washed for 5 times with 200 μL/well PBST; a TMB chromogenic solution (BD, 55214) was added at 100 μL/well for color development at 37° C. for 8 min. 2 M HCl stop solution was added at 100 μL/well. After the addition of the stop solution, the 450 nm values were read using a microplate reader within 30 min. Data were analyzed using GraphPad Prism 6.0 software and avidity fitting was performed to give EC50 values. The results are shown in Table 3.

TABLE 3
Avidity of heavy chain single-domain antibodies for TGFβ protein

| | Avidity EC50 (nM) | | |
|---|---|---|---|
| Antibody | TGFβ1 | TGFβ2 | TGFβ3 |
| B2A1 | 3.824 | 1.071 | 1.254 |
| B2A9 | 5.427 | 1.255 | 1.459 |
| B2C9 | 4.83 | 1.415 | 1.213 |
| B1B8 | 0.5332 | 0.4644 | 0.817 |
| B1F8 | 0.7852 | 1.422 | 1.288 |
| B3D6 | 0.7471 | 0.9394 | 1.22 |
| B2G8 | 1.723 | 1.731 | 1.471 |
| B2C2 | 1.248 | 1.423 | 1.433 |
| B1B7 | 7.006 | 148.5 | 162 |
| B1C5 | 0.442 | 1.85 | 0.5459 |
| B2F6 | NA | 0.7742 | 1.029 |
| B2H1 | 130.3 | 1.542 | 1.713 |
| B2A2 | 48 | 1.178 | 1.296 |
| B3D2 | 69.15 | 27.64 | 1.188 |
| B3C5 | NA | 3.518 | 1.352 |
| B3F3 | 1.48 | 1.625 | 1.464 |
| B3F1 | NA | 148.2 | 1.335 |

### Example 5: Inhibition of Biological Activity of TGF-β1/2/3 by the Antibody

The antibody molecule affected the proliferation of TF-1 cells by inhibiting TGF-β1/2/3. The number of cells was detected using chemiluminescence. TF-1 cells (ATCC) cultured in 1640 medium containing 10% FBS and 2 ng/mL rhGM-CSF (R&D, Cat. No. 7954-GM-050/CF) were inoculated at 1000/well to a 96-well culture plate. TGF-β 1/2/3 protein was added at a concentration of 1 ng/mL per well, and then the gradiently diluted antibody samples to be tested were added separately. The dilution was performed 20-fold in sequence with the highest concentration of 100 nM for a total of 3 concentration gradients. Negative control (only TGF-β was added, and no antibody was added) and blank control groups (only TF-1 cells, and no TGF-β or antibody was added) were set at the same time. The mixture was cultured in an incubator for 120 h, 80 μL of Cell Counting-Lite 2.0 Luminescent Cell Viability Assay (purchased from Vazyme, DD1101) reagent was added per well for 10-15 min of reaction, and the absorbance at OD450 nm was detected with the LUX multifunctional microplate reader. Data were analyzed using GraphPad Prism 6.0 software to compare the inhibitory effect of the antibodies on the biological activity of TGF-β on TF-1 cells.

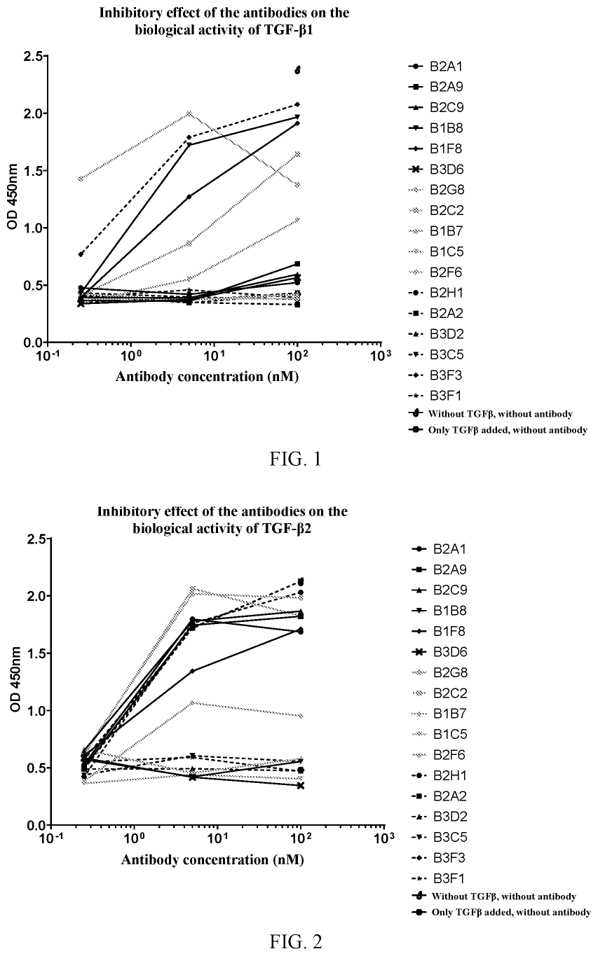

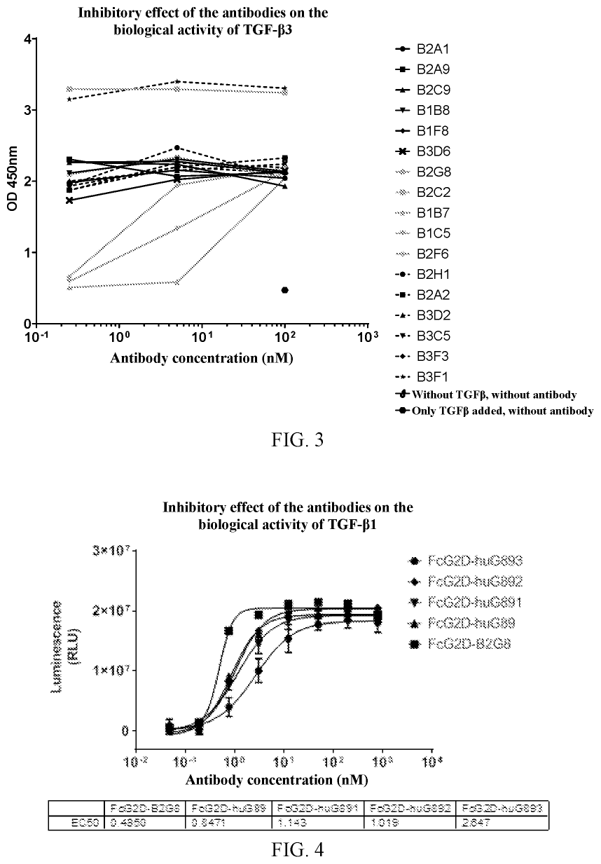

The results are shown in FIG. 1, FIG. 2 and FIG. 3. B2G8, B3F3 and B1B8 had better TGFβ1 inhibitory activity than other candidate antibodies, and also had inhibitory effects on biological activity of TGF-β2/3. Combining the results of the avidity of the antibodies to bind to TGF-β in Example 2, antibodies B1B8, B2G8 and B3F3 have high avidity for TGF-β1 and strong biological inhibitory activity against TGF-β1, while having cross inhibitory activity against TGF-β2/3.

### Example 6: Construction and Preparation of Humanized Heavy Chain Single-Domain Antibody and Bivalent Heavy-Chain Antibody

To humanize B2G8, the primary method involved using the human IgG germline IGHV3-23*01 as a template for CDR (complementarity determining region) grafting, transplanting the Vicugna pacos CDR sequence onto the human heavy chain sequence, and performing a back mutation on the amino acid in the framework region to obtain humanized sequences set forth in SEQ ID NOs: 20-23. Amino acid S marked by a box in the CDR2 of the humanized sequences set forth in SEQ TD NOs: 21-23 was mutated into amino acid A. The FcG2D heavy-chain antibody was constructed by ligating the encoding DNA sequence of the heavy chain variable region of the genetically synthesized monoclonal antibodies to the engineered Fc encoding DNA to obtain an anti-TGFβ3 antibody expression plasmid, typically using pcDNA3.1(−) (purchased from Invitrogen) or other eukaryotic expression vectors as the vectors. Sequence information of the parent antibody (FcG2D-B2G8) and the humanized heavy-chain antibodies (FcG2D-huG89, FcG2D-huG891, FcG2D-huG892 and FcG2D-huG893) is shown in Table 4. Humanized antibodies of other heavy chain single-domain antibodies (including B1B8-Fc, B3F3-Fc, etc.) were also designed. In addition, bivalent heavy-chain antibodies huG892-Fc-huG892 and B1B8-Fc-B1B8 were also constructed. The specific sequences are shown in Table 4. The sequences of other bivalent heavy-chain antibodies (including huG891-Fc-huG891, huG893-Fc-huG893, B2G8-Fc-B2G8, B3F3-Fc-B33F3, etc.) were also designed.

TABLE 4
Sequence information of parent antibody and humanized heavy-chain antibody
(numbering scheme IMGT)

| | | Amino acid sequence (the underlined parts represent CDR regions, | SEQ ID |
|---|---|---|---|
| Antibody | Domain | and parts within boxes represent mutation sites) | NO |
| FcG2D- | Fc constant | DKTHTCPPCPAPPVAGPSVFLFPPKPKDTLMISRTPEVTCVVVDVSH | 18 |
| B2G8 | region | EAPEVQFNWYVDGVEVHNAKTKPREEQFNSTFRVVSVLTVVHQDWLN | |
| | | GKEYKCKVSNKGLPAPIEKTISKTKGQPREPQVYTLPPSRDELTKNQ | |
| | | VSLTCLVKGFYPSDIAVEWESNGQPENNYKTTPPVLDSDGSFFLYSK | |
| | | LTVDKSRWQQGNVFSCSVMHEALHNHYTQKSLSLSPG | |
| | Linker peptide | GGGGS | 19 |
| | Single domain | QVQLVESGGGLVQPGGSLRLSCAASGISFSYYDMGWFRQAPGKOREL | 7 |
| | heavy chain | VAIVTSGGRTNYADSVKGRFTISRETAKSTMYLQMNSLKSEDTGVYY | |
| | variable region | CNAHWIVTTDYWGQGTQVTVAS | |
| FcG2D- | Fc constant | DKTHTCPPCPAPPVAGPSVFLFPPKPKDTLMISRTPEVTCVVVDVSH | 18 |
| huG89 | region | EAPEVQFNWYVDGVEVHNAKTKPREEQFNSTFRVVSVLTVVHQDWLN | |
| | | GKEYKCKVSNKGLPAPIEKTISKTKGQPREPQVYTLPPSRDELTKNQ | |
| | | VSLTCLVKGFYPSDIAVEWESNGQPENNYKTTPPVLDSDGSFFLYSK | |
| | | LTVDKSRWQQGNVFSCSVMHEALHNHYTQKSLSLSPG | |
| | Linker peptide | GGGGS | 19 |
| | Single domain | EVOLLESGGGLVQPGGSLRLSCAASGISFSYYDMGWFRQAPGKOREL | 20 |
| | heavy chain | VSIVTSGGRTNYADSVKGRFTISRDNSKSTLYLOMNSLRAEDTGVYY | |
| | variable region | CNAHWIVTTDYWGQGTLVTVSS | |
| FcG2D- | Fc constant | DKTHTCPPCPAPPVAGPSVFLFPPKPKDTLMISRTPEVTCVVVDVSH | 18 |
| huG891 | region | EAPEVQFNWYVDGVEVHNAKTKPREEQFNSTFRVVSVLTVVHQDWLN | |
| | | GKEYKCKVSNKGLPAPIEKTISKTKGQPREPQVYTLPPSRDELTKNQ | |
| | | VSLTCLVKGFYPSDIAVEWESNGQPENNYKTTPPVLDSDGSFFLYSK | |
| | | LTVDKSRWQQGNVFSCSVMHEALHNHYTQKSLSLSPG | |
| | Linker peptide | GGGGS | 19 |
| | Single domain | EVQLLESGGGLVQPGGSLRLSCAASGISFSYYDMGWFRQAPGKOREL | 21 |
| | heavy chain | 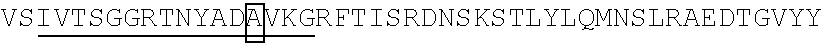 | |
| | variable region | CNAHWIVTTDYWGQGTLVTVSS | |
| FcG2D- | Fc constant | DKTHTCPPCPAPPVAGPSVFLFPPKPKDTLMISRTPEVTCVVVDVSH | 18 |
| huG892 | region | EAPEVQFNWYVDGVEVHNAKTKPREEQFNSTFRVVSVLTVVHQDWLN | |
| | | GKEYKCKVSNKGLPAPIEKTISKTKGQPRE PQVYTLPPSRDELTKNQ | |
| | | VSLTCLVKGFYPSDIAVEWESNGQPENNYKTTPPVLDSDGSFFLYSK | |
| | | LTVDKSRWQQGNVFSCSVMHEALHNHYTQKSLSLSPG | |
| | Linker peptide | GGGGS | 19 |
| | Single domain | EVQLLESGGGLVQPGGSLRLSCAASGISFSYYDMGWFRQAPGKOREL | 22 |
| | heavy chain | 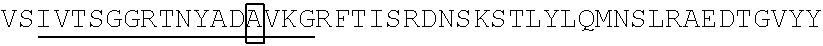 | |
| | variable region | CNAHWIVTTDYWGQGTLVTVSS | |
| FcG2D- | Fc constant | DKTHTCPPCPAPPVAGPSVFLFPPKPKDTLMISRTPEVTCVVVDVSH | 18 |
| huG893 | region | EAPEVQFNWYVDGVEVHNAKTKPREEQFNSTFRVVSVLTVVHODWLN | |
| | | GKEYKCKVSNKGLPAPIEKTISKTKGQPREPQVYTLPPSRDELTKNQ | |
| | | VSLTCLVKGFYPSDIAVEWESNGQPENNYKTTPPVLDSDGSFFLYSK | |
| | | LTVDKSRWQQGNVFSCSVMHEALHNHYTQKSLSLSPG | |
| | Linker peptide | GGGGS | 19 |
| | Single domain | EVQLLESGGGLVQPGGSLRLSCAASGISFSYYDMGWERQAPGKOREL | 23 |
| | heavy chain | 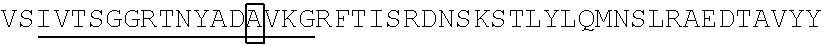 | |
| | variable region | CNAHWIVTTDYWGQGTLVTVSS | |
| huG892- | Single domain | EVQLLESGGGLVQPGGSLRLSCAASGISFSYYDMGWERQAPGKOREL | 22 |
| Fc- | heavy chain | 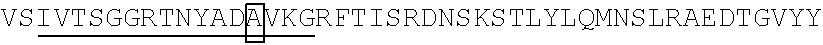 | |
| huG892 | variable region | CNAHWIVTTDYWGQGTLVTVSS | |
| | Linker peptide | GGGGS | 19 |
| | Fc constant | DKTHTCPPCPAPPVAGPSVFLFPPKPKDTLMISRTPEVTCVVVDVSH | 18 |
| | region | EAPEVQFNWYVDGVEVHNAKTKPREEQFNSTFRVVSVLTVVHQDWLN | |
| | | GKEYKCKVSNKGLPAPIEKTISKTKGQPREPQVYTLPPSRDELTKNQ | |
| | | VSLTCLVKGFYPSDIAVEWESNGQPENNYKTTPPVLDSDGSFFLYSK | |
| | | LTVDKSRWQQGNVFSCSVMHEALHNHYTQKSLSLSPG | |
| | Linker peptide | GGGGS | 19 |
| | Single domain | EVQLLESGGGLVQPGGSLRLSCAASGISFSYYDMGWFRQAPGKOREL | 22 |
| | heavy chain | 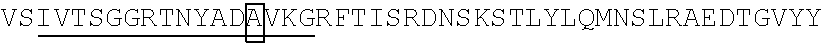 | |
| | variable region | CNAHWIVTTDYWGQGTLVTVSS | |
| B1B8-Fc- | Single domain | EVQVVESGGGLVQPGGSLTLSCVASGFAFSFTPMRWVRQAPGKGLEW | 4 |
| B1B8 | heavy chain | VSSISIRGDTTDYADSVKGRFTISRDNAKNTLYLQMNSLKPEDTAVY | |
| | variable region | YCSRGSSISTSTERTQGTQVTVSS | |
| | Linker peptide | GGGGS | 19 |
| | Fc constant | DKTHTCPPCPAPPVAGPSVFLFPPKPKDTLMISRTPEVTCVVVDVSH | 18 |
| | region | EAPEVQFNWYVDGVEVHNAKTKPREEQFNSTFRVVSVLTVVHQDWLN | |
| | | GKEYKCKVSNKGLPAPIEKTISKTKGQPREPQVYTLPPSRDELTKNQ | |
| | | VSLTCLVKGFYPSDIAVEWESNGQPENNYKTTPPVLDSDGSFFLYSK | |
| | | LTVDKSRWQQGNVFSCSVMHEALHNHYTQKSLSLSPG | |
| | Linker peptide | GGGGS | 19 |
| | Single domain | EVQVVESGGGLVQPGGSLTLSCVASGFAFSFTPMRWVRQAPGKGLEW | 4 |
| | heavy chain | VSSISIRGDTTDYADSVKGRFTISRDNAKNTLYLQMNSLKPEDTAVY | |
| | variable region | YCSRGSSISTSTERTQGTQVTVSS | |

Plasmid extraction was performed using EndoFree Plasmid Giga Kit (Qiagen, Cat. No. 12391), following the manufacturer's manual. CHO-S cells were cultured in CD CHO medium (Gibco, Cat. No. 10743-029) in a 37° C., 5% CO₂ incubator according to the manufacturer's manual. After the cells were prepared, CHO-S cells were co-transfected with plasmids containing heavy chain sequences to express an antibody. The day after transfection, the culture temperature was adjusted to 32° C. and 3.5% 2×EFC+(Gibco, Cat. No. A2503105) was added daily. After 14 days of culture, expression supernatants were harvested by centrifugation at 800×g. The supernatants were filtered through a 0.22 μm filter membrane, and purified by protein A affinity chromatography and cation exchange chromatography to give the antibodies in the culture supernatants. The concentration of the purified antibody was determined by UV absorbance at 280 nm and the corresponding extinction coefficient for each protein. The purity and homogeneity of the antibodies were assessed by SDS-PAGE and SE-HPLC. The antibodies were further purified by a second purification through ion exchange and SEC with Superdex 200 to prepare antibody samples of higher purities for later use.

### Example 7: Assessment of Stability in Acidic Condition and Thermal Condition for Antibodies

Antibodies in Table 4 were assessed for stability in acidic condition and thermal condition according to the following methods. When the antibody molecules were subjected to protein A affinity chromatography, the eluted antibody solution was not neutralized in the acid elution step (using a citric acid buffer at pH 3.5). After maintenance for a period of time in this buffer, 1/10 volume of 1 M Tris-HCl (pH 8.0) was added at the 30th min for neutralization and HPLC-SEC detection was performed on this sample. As shown in Table 5, the humanized antibody molecules did not aggregate or degrade after 30 min of treatment at pH 3.5, and the purity changed by less than 4%, indicating that the humanized antibody molecules can maintain stability in acidic environments. Meanwhile, HPLC-SEC detection of the sample was performed after incubation at 40° C. for 14 days in an incubator. No aggregation or degradation was found and the purity changed by less than 4%, indicating that it can maintain stability in an environment at 40° C. As shown in Table 6, similar results were obtained in acid stability and thermal stability experiments for other humanized antibodies and bivalent heavy-chain antibodies (including huB1B8-Fc, huB3F3-Fc, huG891-Fc-huG891, huG893-Fc-huG893, B2G8-Fc-B2G8, B3F3-Fc-B3F3, etc.).

TABLE 5
Acid stability assessment of antibodies

pH 3.5, 30 min

| | Pre-treatment | Post-treatment |
|---|---|---|
| Sample | purity by SEC | purity by SEC |
| FcG2D-B2G8 | 93.88% | 93.78% |
| FcG2D-huG89 | 97.47% | 97.28% |
| FcG2D-huG891 | 97.47% | 97.08% |
| FcG2D-huG892 | 97.06% | 96.96% |
| FcG2D-huG893 | 97.02% | 96.94% |
| huG892-Fc-huG892 | 98.36% | 98.06% |
| B1B8-Fc-B1B8 | 99.64% | 98.99% |

TABLE 6
Thermal stability assessment of antibodies

| | Thermal stability test at 40° C. | | |
|---|---|---|---|
| | Purity by | Purity by | Purity by |
| Sample | SEC on Day 0 | SEC on Day 7 | SEC on Day 14 |
| FcG2D-B2G8 | 92.08% | 89.99% | 91.74% |
| FcG2D-huG89 | 97.16% | 95.76% | 93.41% |
| FcG2D-huG891 | 96.68% | 96.38% | 93.76% |
| FcG2D-huG892 | 96.77% | 96.04% | 93.65% |
| FcG2D-huG893 | 96.72% | 96.02% | 93.51% |
| huG892-Fc-huG892 | 98.08% | 97.77% | 97.15% |
| B1B8-Fc-B1B8 | 98.25% | 96.22% | 95.36% |

The results show that the humanized molecules FcG2D-hu89, FcG2D-hu891, FcG2D-hu892, FcG2D-hu893, huG892-Fc-huG892, and B1B8-Fc-B1B8 all had good acid stability and thermal stability. Also, after purification by protein A, the purities of the antibodies were all more than 960%, showing good expression stability.

### Example 8: Inhibition of Biological Activity of TGF-β

The antibody molecule affected the proliferation of TF-1 cells by inhibiting TGF-β. The number of cells was detected using chemiluminescence. TF-1 cells (ATCC) cultured in 1640 medium containing 10% FBS and 2 ng/mL rhGM-CSF were inoculated at 6000/well to a 96-well culture plate. TGF-β1 protein was added at a concentration of 16 ng/mL per well, and then the gradiently diluted antibody samples to be tested were added separately. The dilution was performed 4-fold in sequence for a total of 8 concentration gradients. The mixture was cultured in an incubator for 120 h, 80 μL of Cell Counting-Lite 2.0 Luminescent Cell Viability Assay (purchased from Vazyme, DD1101) reagent was added per well for 10-15 min of reaction, and the chemiluminescence value was read with the LUX multifunctional microplate reader. Data were analyzed using GraphPad Prism 6.0 software. With the final antibody concentration as the abscissa and the chemiluminescence value (experimental well reading−negative control well reading) as the ordinate, a four-parameter non-linear regression was performed to fit the dose-response curve and calculate the median effect concentration (EC50) for the test sample and the reference sample. The inhibitory effect of the antibodies on the biological activity of TGF-β1 on TF-1 cells was compared.

The results are shown in FIG. 4. The EC50 values of FcG2D-huG89, FcG2D-huG891 and FcG2D-huG892 were within one fold of that of the parent FcG2D-B2G8, which shows that the 3 humanized molecules have strong inhibitory activity. The same method was used to assess huG892-Fc-huG892 and B1B8-Fc-B1B8 for TGF-β1 inhibitory activity, with EC50 values of 0.424 nM and 0.813 nM, respectively, both having strong activity. Similar results were obtained for other humanized antibodies and bivalent heavy-chain antibodies (including huB1B8-Fc, huB3F3-Fc, huG891-Fc-huG891, huG893-Fc-huG893, B2G8-Fc-B2G8, B3F3-Fc-B3F3, etc.).

### Example 9: Construction of Bispecific Antibody and Use

### 9.1 Construction of Bispecific Antibody

Reverse translation of DNA code and synthesis of DNA fragments were performed according to the amino acid sequence of the bispecific antibody shown in Table 7 (Genecreate), which was constructed into expression vector pcDNA3.1, and transiently transfected into 293 or CHO cells for expression. The supernatant was collected for protein purification to obtain a bispecific antibody with the purity of not less than 95%. Specific vector construction, transient transfection and protein purification methods are found in the preceding examples. The structure of the bispecific antibody is: comprising a light chain and a heavy chain, wherein the light and heavy chain pair and form an interchain disulfide bond, and the two heavy chains pair and form an interchain disulfide bond, wherein the heavy chain is (VH)-(CH1)-(hinge region)-(Fc)-(linker peptide)-(VHH), the light chain is (VL)-(light chain constant region), VH and VL are the heavy chain and light chain variable regions of an anti-VEGF antibody, and VHH is the heavy chain variable region of an anti-TGF-β3 single-domain antibody.

TABLE 7
Amino acid sequence of bispecific antibody (number scheme IMGT)

| Bispecific | | | Amino acid sequence (the underlined | SEQ ID |
|---|---|---|---|---|
| antibody | Chain | Domain | parts represent CDR regions) | NO: |
| G6-Fc- | Heavy | Anti-VEGF | EVQLVESGGGLVQPGGSLRLSCAASGFTISDYWIHWVRQ | 24 |
| huG892 | chain | heavy chain | APGKGLEWVAGITPAGGYTYYADSVKGRFTISADTSKNT | |
| | | variable | AYLQMNSLRAEDTAVYYCARFVFFLPYAMDYWGQGTLVT | |
| | | region (VH) | VSS | |
| | | | HCDR1: DYWIH (SEQ ID NO: 82) | |
| | | | HCDR2: GITPAGGYTYYADSVKG (SEQ ID NO: 83) | |
| | | | HCDR3: FVFFLPYAMDY (SEQ ID NO: 84) | |
| | | CH1 | ASTKGPSVFPLAPSSKSTSGGTAALGCLVKDYFPEPVTV | 25 |
| | | | SWNSGALTSGVHTFPAVLQSSGLYSLSSVVTVPSSSLGT | |
| | | | QTYICNVNHKPSNTKVDKKVEPKSC | |
| | | Hinge region | DKTHTCP | 26 |
| | | Fc constant | DKTHTCPPCPAPPVAGPSVFLFPPKPKDTLMISRTPEVT | 18 |
| | | region | CVVVDVSHEAPEVQFNWYVDGVEVHNAKTKPREEQENST | |
| | | | FRVVSVLTVVHQDWLNGKEYKCKVSNKGLPAPIEKTISK | |
| | | | TKGQPREPQVYTLPPSRDELTKNQVSLTCLVKGFYPSDI | |
| | | | AVEWESNGQPENNYKTTPPVLDSDGSFFLYSKLTVDKSR | |
| | | | WQQGNVFSCSVMHEALHNHYTQKSLSLSPG | |
| | | Linker | GGGGS | 19 |
| | | peptide | | |
| | | Anti-TGF-β | EVQLLESGGGLVQPGGSLRLSCAASGISFSYYDMGWERQ | 22 |
| | | single | APGKQRELVSIVTSGGRTNYADAVKGRFTISRDNSKNTL | |
| | | domain | YLQMNSLRAEDTGVYYCNAHWIVTTDYWGQGTLVTVSS | |
| | | heavy chain | | |
| | | variable | | |
| | | region | | |
| | | (VHH) | | |
| | Light | Anti-VEGF | DIQMTQSPSSLSASVGDRVTITCRASQDVSTAVAWYQQK | 27 |
| | chain | light chain | PGKAPKLLIYSASFLYSGVPSRESGSGSGTDFTLTISSL | |
| | | variable | QPEDFATYYCQQGYGNPPTFGQGTKVEIK | |
| | | region (VL) | LCDR1: RASQDVSTAVA (SEQ ID NO: 85) | |
| | | | LCDR2: SASFLYS (SEQ ID NO: 86) | |
| | | | LCDR3: QQGYGNPPT (SEQ ID NO: 87) | |
| | | Light chain | RTVAAPSVFIFPPSDEQLKSGTASVVCLLNNFYPREAKV | 28 |
| | | constant | QWKVDNALQSGNSQESVTEQDSKDSTYSLSSTLTLSKAD | |
| | | region | YEKHKVYACEVTHQGLSSPVTKSENRGEC | |
| Beva-Fc- | Heavy | Anti-VEGF | EVQLVESGGGLVQPGGSLRLSCAASGYTFTNYGMNWVRQ | 29 |
| huG892 | chain | heavy chain | APGKGLEWVGWINTYTGEPTYAADFKRRFTFSLDTSKST | |
| | | variable | AYLQMNSLRAEDTAVYYCAKYPHYYGSSHWYFDVWGQGT | |
| | | region | LVTVSS | |
| | | | HCDR1: GYTFTNYGMN (SEQ ID NO: 88) | |
| | | | HCDR2: WINTYTGEPTYAADFKR (SEQ ID NO: 89) | |
| | | | HCDR3: YPHYYGSSHWYFDV (SEQ ID NO: 90) | |
| | | CH1 | ASTKGPSVFPLAPSSKSTSGGTAALGCLVKDYFPEPVTV | 25 |
| | | | SWNSGALTSGVHTFPAVLQSSGLYSLSSVVTVPSSSLGT | |
| | | | QTYICNVNHKPSNTKVDKKVEPKSC | |
| | | Hinge region | DKTHTCP | 26 |
| | | Fc constant | DKTHTCPPCPAPPVAGPSVFLFPPKPKDTLMISRTPEVT | 18 |
| | | region | CVVVDVSHEAPEVQFNWYVDGVEVHNAKTKPREEQENST | |
| | | | FRVVSVLTVVHQDWLNGKEYKCKVSNKGLPAPIEKTISK | |
| | | | TKGQPREPQVYTLPPSRDELTKNQVSLTCLVKGFYPSDI | |
| | | | AVEWESNGQPENNYKTTPPVLDSDGSFFLYSKLTVDKSR | |
| | | | WQQGNVFSCSVMHEALHNHYTQKSLSLSPG | |
| | | Linker | GGGGS | 19 |
| | | peptide | | |
| | | Anti-TGF-β | EVQLLESGGGLVQPGGSLRLSCAASGISFSYYDMGWERQ | 22 |
| | | single | APGKQRELVSIVTSGGRTNYADAVKGRFTISRDNSKNTL | |
| | | domain | YLQMNSLRAEDTGVYYCNAHWIVTTDYWGQGTLVTVSS | |
| | | heavy chain | | |
| | | variable | | |
| | | region | | |
| | Light | Anti-VEGF | DIQMTQSPSSLSASVGDRVTITCSASQDISNYLNWYQQK | 30 |
| | chain | light chain | PGKAPKVLIYFTSSLHSGVPSRFSGSGSGTDFTLTISSL | |
| | | variable | QPEDFATYYCQQYSTVPWTFGQGTKVEIK | |
| | | region | LCDR1: SASQDISNYLN (SEQ ID NO: 91) | |
| | | | LCDR2: FTSSLHS (SEQ ID NO: 92) | |
| | | | LCDR3: QQYSTVPWT (SEQ ID NO: 93) | |
| | | Light chain | RTVAAPSVFIFPPSDEQLKSGTASVVCLLNNFYPREAKV | 28 |
| | | constant | QWKVDNALQSGNSQESVTEQDSKDSTYSLSSTLTLSKAD | |
| | | region | YEKHKVYACEVTHQGLSSPVTKSENRGEC | |

### 9.2 Study on the Stability of Bispecific Antibody

The two prepared bispecific antibodies G6-Fc-huG892 and Beva-Fc-huG892 were subjected to thermal accelerated stability study. HPLC-SEC detection of the sample was performed after incubation at 40° C. for 14 days in an incubator. No aggregation or degradation was found and the purity changed by less than 400, indicating that it can maintain stability in an environment at 40° C. The results are shown in Table 8. The results indicate that the stability of both bispecific antibodies is good.

TABLE 8
Thermal accelerated stability study
of bispecific antibodies at 40° C.

| | | Purity | | Purity |
|---|---|---|---|---|
| Bispecific | Concentration | on | Concentration | on |
| antibody | on day 0 | day 0 | on day 14 | day 14 |
| G6-Fc-huG892 | 6.3 mg/mL | 99.45% | 6.3 mg/mL | 98.92% |
| Beva-Fc-huG892 | 4.8 mg/mL | 99.42% | 4.8 mg/mL | 99.26% |

### 9.3 In Vivo Anti-Tumor Efficacy Assessment of Bispecific Antibody

G6-Fc-huG892 can bind to human or murine VEGF and human or murine TGF-β. Beva-Fc-huG892 can bind to human VEGF and human or murine TGF-β.

### \textcircled{1} BALB/c Wild-Type Mouse Syngeneic Subcutaneously Transplanted Tumor Model

In the CT26 and EMT-6 subcutaneous tumor models of wild-type mice, the anti-tumor efficacy of the antibodies was assessed by intraperitoneal administration of G6-Fc-huG892 and monitoring of tumor size.

### a). Experimental Materials:

Cell: CT26 (mouse colon cancer cell line, purchased from ATCC) and EMT-6 (mouse breast cancer cell line, purchased from ATCC);
Mouse: BALB/c wild-type mice, female, purchased from Beijing Vital River;
Inoculation mode: CT26 cells and EMT-6 cells were cultured, collected, adjusted for cell concentration, and subcutaneously inoculated on the dorsum at 1×10⁶ cells/mouse (0.1 mL/mouse), respectively. When the tumor was grown to 100-200 mm³, the mice were grouped for administration, 6 mice in each group and two groups for each kind of cells;
Test drug: G6-Fc-huG892.
Negative control: normal saline;

Administration mode: when the tumor was grown to 100-200 mm³, intraperitoneal administration was carried out at a dose of 10 mg/kg. The intraperitoneal administration was carried out 3 times a week for 2 weeks, and the initial administration was day 0.

Tumor volume: tumor length and width were measured 3 times a week. One group would be terminated when tumor volume of the group approached 2000 mm³ or a single mouse tumor volume reached 3000 mm³.

### b). Experimental Results

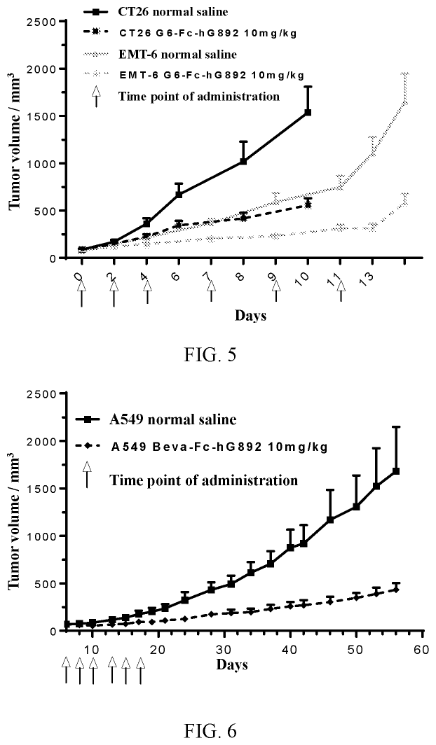

The in vivo efficacy experimental results of G6-Fc-huG892 are shown in FIG. 5. In the CT-26 subcutaneous tumor model and the EMT-6 subcutaneous tumor model, G6-Fc-huG892 both had a remarkable tumor inhibition effect.

### \textcircled{2} Balb/c-Nude Mouse Subcutaneous Xenograft Tumor Model

In the human lung cancer A549 subcutaneous tumor model of balb/c-nude mice, the anti-tumor efficacy of the antibodies was assessed by intraperitoneal administration of Beva-Fc-huG892 and monitoring of tumor size.

### a). Experimental Materials:

Cell: A549 (human lung cancer cell strain, purchased from ATCC);
Mouse: balb/c-nude mice, female, purchased from Beijing Vital River;
Inoculation mode: A549 cells were cultured, collected, adjusted for cell concentration, and subcutaneously inoculated on the dorsum at 5×10⁶ cells/mouse (0.1 mL/mouse). When the tumor was grown to 100-200 mm³, the mice were grouped for administration, 6 mice in each group and 2 groups in total;
Test drug: Beva-Fc-huG892.
Negative control: normal saline;

Administration mode: when the tumor was grown to 100-200 mm³, intraperitoneal administration was carried out. The bispecific antibody was administered at a dose of 10 mg/kg. The intraperitoneal administration was carried out separately 3 times a week for 2 weeks, and the initial administration was day 0.

Tumor volume: tumor length and width were measured 3 times a week. One group would be terminated when tumor volume of the group approached 2000 mm³ or a single mouse tumor volume reached 3000 mm³.

### b). Experimental Results

The in vivo efficacy experimental results of Beva-Fc-huG892 are shown in FIG. 6. In the A549 subcutaneous tumor model, Beva-Fc-huG892 had a remarkable tumor inhibition effect.

### \textcircled{3} Assessment of Anti-VEGF In Vitro Biological Activity of Bispecific Antibodies

The antibody molecule affected the proliferation of HUVEC cells by inhibiting VEGF. The number of cells was detected using chemiluminescence, and the inhibition rate was calculated. HUVEC cells (purchased from AllCells) cultured in complete endothelial cell medium were inoculated at 5000/well to a 96-well culture plate and cultured overnight; the next day, the culture medium was discarded, 200 ng/mL of VEGFA protein (purchased from GeneScript, Z02689) was added to each well, and then the gradiently diluted antibody samples to be tested Beva-Fc-hG892 and G6-Fc-hG892 were added separately. The dilution was performed 3-fold in sequence with the highest concentration of 300 μg/mL, and a total of 9-11 concentration gradients were set. The mixture was cultured in an incubator for 3 days. On day 3, an equal volume of 100 μL of Cell Counting-Lite 2.0 Luminescent Cell Viability Assay (purchased from Vazyme, DD1101) reagent was added per well for 10-15 min of reaction, and the absorbance at OD450 nm was detected with the LUX multifunctional microplate reader. Data were analyzed using GraphPad Prism 6.0 software, and the median effect concentration (EC50) of the antibody to inhibit VEGFA-induced HUVEC cell proliferation effect was calculated.

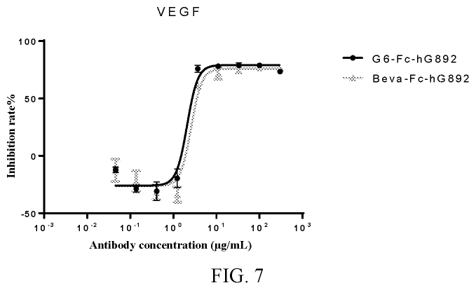

The results are shown in FIG. 7 and Table 9. The inhibitory activity of both bispecific antibody molecules on VEGF was consistent.

TABLE 9
Median effect concentration (EC50) of antibody
to inhibit biological activity of VEGF

| Antibody | EC50(μg/mL) |
|---|---|
| G6-Fc-hG892 | 2.090 |
| Beva-Fc-hG892 | 2.602 |

### \textcircled{4} Assessment of Anti-TGFβ 1/2/3 In Vitro Biological Activity of Bispecific Antibodies

The antibody molecule affected the proliferation of TF-1 cells by inhibiting TGF-β. The number of cells was detected using chemiluminescence. TF-1 cells (ATCC) cultured in 1640 medium containing 10% FBS and 2 ng/mL rhGM-CSF were inoculated at 1000/well to a 96-well culture plate. TGF-β1 (GenScript, Z03411) or TGF-β2 (GenScript, Z03429) or TGF-β3 (GenScript, Z03430) protein was added at a concentration of 1 ng/mL per well, and then the gradiently diluted antibody samples to be tested Beva-Fc-hG892 and G6-Fc-hG892 were added separately. The dilution was performed 5-fold in sequence with the highest concentration of 100 nM, and a total of 9-11 concentration gradients and 3 replicate wells were set. The mixture was cultured in an incubator for 120 h, 80 μL of Cell Counting-Lite 2.0 Luminescent Cell Viability Assay (purchased from Vazyme, DD1101) reagent was added per well for 10-15 min of reaction, and the absorbance at OD450 nm was detected with the LUX multifunctional microplate reader. Data were analyzed using GraphPad Prism 6.0 software, and the EC50 value for the blocking effect of antibodies on TGF-β-induced TF-1 cell proliferation inhibition was calculated.

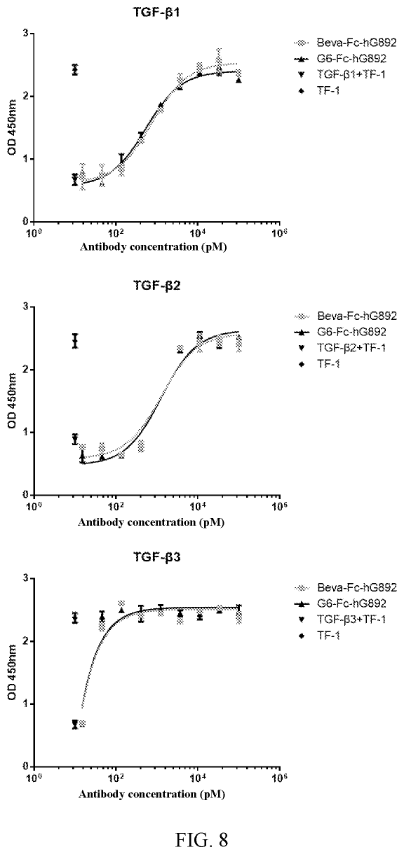

The results are shown in FIG. 8 and Table 10. The inhibitory activity of both bispecific antibody molecules on TGF-β1/2/3 was consistent.

TABLE 10
Median effect concentration (EC50) of antibody
to inhibit biological activity of TGF-β

| | TGF-β1 | TGF-β2 | TGF-β3 |
|---|---|---|---|
| Antibody | EC50 (pM) | EC50 (pM) | EC50 (pM) |
| G6-Fc-hG892 | 778.2 | 1299 | 0.002 |
| Beva-Fc-hG892 | 540.3 | 1281 | 0.002 |

All documents mentioned in the present invention are incorporated by reference, just as each document is cited separately as a reference. In addition, it should be understood that various modifications or changes may be made by those skilled in the art after reading the above teachings of the present invention, and these equivalent forms also fall within the scope defined by the claims appended hereto.

1. An anti-TGFβ antibody, comprising or consisting of a sequence selected from the group consisting of:
(1) B2G8, comprising a CDR1, a CDR2, and a CDR3 comprised in a sequence set forth in SEQ ID NO: 7, preferably comprising a CDR1 set forth in SEQ ID NO: 49, a CDR2 set forth in SEQ ID NO: 50, and a CDR3 set forth in SEQ ID NO: 51, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 49, the CDR2 set forth in SEQ ID NO: 50, and/or the CDR3 set forth in SEQ ID NO: 51 and retaining the binding affinity to TGFβ, preferably comprising the CDR1 set forth in SEQ ID NO: 49, a variant of the CDR2 set forth in SEQ ID NO: 50, and the CDR3 set forth in SEQ ID NO: 51, wherein the variant of the CDR2 set forth in SEQ ID NO: 50 is that the 13th amino acid of an amino acid sequence set forth in SEQ ID NO: 50 is replaced with alanine, or
comprising a CDR1 set forth in SEQ ID NO: 49, a CDR2 set forth in SEQ ID NO: 50, and a CDR3 set forth in SEQ ID NO: 51, wherein amino acids 5-8 in the CDR1 set forth in SEQ ID NO: 49, amino acids 1-5 and amino acids 7-9 in the CDR2 set forth in SEQ ID NO: 50, and amino acid 1 and amino acids 4-9 in the CDR3 set forth in SEQ ID NO: 51 are selected from amino acid X, wherein the amino acid X is selected from the group consisting of Ala, Arg, Asn, Asp, Cys, Gln, Glu, Gly, His, Ile, Leu, Lys, Met, Phe, Pro, Ser, Thr, Trp, Tyr, and Val,
(2) B1B8, comprising a CDR1, a CDR2, and a CDR3 comprised in a sequence set forth in SEQ ID NO: 4, preferably comprising a CDR1 set forth in SEQ ID NO: 40, a CDR2 set forth in SEQ ID NO: 41, and a CDR3 set forth in SEQ ID NO: 42, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 40, the CDR2 set forth in SEQ ID NO: 41, and/or the CDR3 set forth in SEQ ID NO: 42 and retaining the binding affinity to TGFβ, or
comprising a CDR1 set forth in SEQ ID NO: 40, a CDR2 set forth in SEQ ID NO: 41, and a CDR3 set forth in SEQ ID NO: 42, wherein amino acid 3 and amino acids 5-8 in the CDR1 set forth in SEQ ID NO: 40, amino acids 1, 3, and 5-10 in the CDR2 set forth in SEQ ID NO: 41, and amino acids 4-9 and 11 in the CDR3 set forth in SEQ ID NO: 42 are selected from amino acid X, wherein the amino acid X is selected from the group consisting of Ala, Arg, Asn, Asp, Cys, Gln, Glu, Gly, His, Ile, Leu, Lys, Met, Phe, Pro, Ser, Thr, Trp, Tyr, and Val,
(3) B3F3, comprising a CDR1, a CDR2, and a CDR3 comprised in a sequence set forth in SEQ ID NO: 16, preferably comprising a CDR1 set forth in SEQ ID NO: 76, a CDR2 set forth in SEQ ID NO: 77, and a CDR3 set forth in SEQ ID NO: 78, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 76, the CDR2 set forth in SEQ ID NO: 77, and/or the CDR3 set forth in SEQ ID NO: 78 and retaining the binding affinity to TGFβ; or
comprising a CDR1 set forth in SEQ ID NO: 76, a CDR2 set forth in SEQ ID NO: 77, and a CDR3 set forth in SEQ ID NO: 78, wherein amino acids 2-3 and amino acids 5-8 in the CDR1 set forth in SEQ ID NO: 76, amino acids 2-4 and amino acid 6 in the CDR2 set forth in SEQ ID NO: 77, and amino acid 1 and amino acids 3-8 in the CDR3 set forth in SEQ ID NO: 78 are selected from amino acid X, wherein the amino acid X is selected from the group consisting of Ala, Arg, Asn, Asp, Cys, Gln, Glu, Gly, His, Ile, Leu, Lys, Met, Phe, Pro, Ser, Thr, Trp, Tyr, and Val,
(4) B2A1, comprising a CDR1, a CDR2, and a CDR3 comprised in a sequence set forth in SEQ ID NO: 1, preferably comprising a CDR1 set forth in SEQ ID NO: 31, a CDR2 set forth in SEQ ID NO: 32, and a CDR3 set forth in SEQ ID NO: 33, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 31, the CDR2 set forth in SEQ ID NO: 32, and/or the CDR3 set forth in SEQ ID NO: 33 and retaining the binding affinity to TGFβ;
(5) B1F8, comprising a CDR1, a CDR2, and a CDR3 comprised in a sequence set forth in SEQ ID NO: 5, preferably comprising a CDR1 set forth in SEQ ID NO: 43, a CDR2 set forth in SEQ ID NO: 44, and a CDR3 set forth in SEQ ID NO: 45, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 43, the CDR2 set forth in SEQ ID NO: 44, and/or the CDR3 set forth in SEQ ID NO: 45 and retaining the binding affinity to TGFβ;
(6) B3D6, comprising a CDR1, a CDR2, and a CDR3 comprised in a sequence set forth in SEQ ID NO: 6, preferably comprising a CDR1 set forth in SEQ ID NO: 46, a CDR2 set forth in SEQ ID NO: 47, and a CDR3 set forth in SEQ ID NO: 48, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 46, the CDR2 set forth in SEQ ID NO: 47, and/or the CDR3 set forth in SEQ ID NO: 48 and retaining the binding affinity to TGFβ;
(7) B2A9, comprising a CDR1, a CDR2, and a CDR3 comprised in a sequence set forth in SEQ ID NO: 2, preferably comprising a CDR1 set forth in SEQ ID NO: 34, a CDR2 set forth in SEQ ID NO: 35, and a CDR3 set forth in SEQ ID NO: 36, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 34, the CDR2 set forth in SEQ ID NO: 35, and/or the CDR3 set forth in SEQ ID NO: 36 and retaining the binding affinity to TGFβ;
(8) B2C2, comprising a CDR1, a CDR2, and a CDR3 comprised in a sequence set forth in SEQ ID NO: 8, preferably comprising a CDR1 set forth in SEQ ID NO: 52, a CDR2 set forth in SEQ ID NO: 53, and a CDR3 set forth in SEQ ID NO: 54, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 52, the CDR2 set forth in SEQ ID NO: 53, and/or the CDR3 set forth in SEQ ID NO: 54 and retaining the binding affinity to TGFβ;
(9) B1B7, comprising a CDR1, a CDR2, and a CDR3 comprised in a sequence set forth in SEQ ID NO: 9, preferably comprising a CDR1 set forth in SEQ ID NO: 55, a CDR2 set forth in SEQ ID NO: 56, and a CDR3 set forth in SEQ ID NO: 57, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 55, the CDR2 set forth in SEQ ID NO: 56, and/or the CDR3 set forth in SEQ ID NO: 57 and retaining the binding affinity to TGFβ;
(10) B1C5, comprising a CDR1, a CDR2, and a CDR3 comprised in a sequence set forth in SEQ ID NO: 10, preferably comprising a CDR1 set forth in SEQ ID NO: 58, a CDR2 set forth in SEQ ID NO: 59, and a CDR3 set forth in SEQ ID NO: 60, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 58, the CDR2 set forth in SEQ ID NO: 59, and/or the CDR3 set forth in SEQ ID NO: 60 and retaining the binding affinity to TGFβ;
(11) B2F6, comprising a CDR1, a CDR2, and a CDR3 comprised in a sequence set forth in SEQ ID NO: 11, preferably comprising a CDR1 set forth in SEQ ID NO: 61, a CDR2 set forth in SEQ ID NO: 62, and a CDR3 set forth in SEQ ID NO: 63, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 61, the CDR2 set forth in SEQ ID NO: 62, and/or the CDR3 set forth in SEQ ID NO: 63 and retaining the binding affinity to TGFβ;
(12) B2H1, comprising a CDR1, a CDR2, and a CDR3 comprised in a sequence set forth in SEQ ID NO: 12, preferably comprising a CDR1 set forth in SEQ ID NO: 64, a CDR2 set forth in SEQ ID NO: 65, and a CDR3 set forth in SEQ ID NO: 66, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 64, the CDR2 set forth in SEQ ID NO: 65, and/or the CDR3 set forth in SEQ ID NO: 66 and retaining the binding affinity to TGFβ;
(13) B2A2, comprising a CDR1, a CDR2, and a CDR3 comprised in a sequence set forth in SEQ ID NO: 13, preferably comprising a CDR1 set forth in SEQ ID NO: 67, a CDR2 set forth in SEQ ID NO: 68, and a CDR3 set forth in SEQ ID NO: 69, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 67, the CDR2 set forth in SEQ ID NO: 68, and/or the CDR3 set forth in SEQ ID NO: 69 and retaining the binding affinity to TGFβ;
(14) B3D2, comprising a CDR1, a CDR2, and a CDR3 comprised in a sequence set forth in SEQ ID NO: 14, preferably comprising a CDR1 set forth in SEQ ID NO: 70, a CDR2 set forth in SEQ ID NO: 71, and a CDR3 set forth in SEQ ID NO: 72, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 70, the CDR2 set forth in SEQ ID NO: 71, and/or the CDR3 set forth in SEQ ID NO: 72 and retaining the binding affinity to TGFβ;
(15) B3C5, comprising a CDR1, a CDR2, and a CDR3 comprised in a sequence set forth in SEQ ID NO: 15, preferably comprising a CDR1 set forth in SEQ ID NO: 73, a CDR2 set forth in SEQ ID NO: 74, and a CDR3 set forth in SEQ ID NO: 75, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 73, the CDR2 set forth in SEQ ID NO: 74, and/or the CDR3 set forth in SEQ ID NO: 75 and retaining the binding affinity to TGFβ;
(16) B2C9, comprising a CDR1, a CDR2, and a CDR3 comprised in a sequence set forth in SEQ ID NO: 3, preferably comprising a CDR1 set forth in SEQ ID NO: 37, a CDR2 set forth in SEQ ID NO: 38, and a CDR3 set forth in SEQ ID NO: 39, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 37, the CDR2 set forth in SEQ ID NO: 38, and/or the CDR3 set forth in SEQ ID NO: 39 and retaining the binding affinity to TGFβ, and
(17) B3F1, comprising a CDR1, a CDR2, and a CDR3 comprised in a sequence set forth in SEQ ID NO: 17, preferably comprising a CDR1 set forth in SEQ ID NO: 79, a CDR2 set forth in SEQ ID NO: 80, and a CDR3 set forth in SEQ ID NO: 81, or a variant thereof according to the Kabat numbering system and the IMGT numbering system, wherein the variant is an amino acid sequence having one or more (preferably 1, 2 or 3) conservative amino acid mutations (preferably substitutions, insertions or deletions) as compared to the CDR1 set forth in SEQ ID NO: 79, the CDR2 set forth in SEQ ID NO: 80, and/or the CDR3 set forth in SEQ ID NO: 81 and retaining the binding affinity to TGFβ.

2. The anti-TGFβ antibody according to claim 1, comprising a sequence selected from SEQ ID NOs: 1-17 or a variant thereof, or consisting of a sequence selected from SEQ ID NOs: 1-17 or a variant thereof, wherein the variant is a sequence having at least 80%, 85%, 90%, 91%, 92%, 93%, 94%, 95%, 96%, 97%, 98%, or 99% homology to the amino acid sequence set forth in the SEQ ID NOs and retaining the binding affinity to TGFβ, or an amino acid sequence having one or more (preferably 1, 2, 3, 4, 5, 6, 7, 8, 9 or 10) conservative amino acid mutations (preferably substitutions, insertions or deletions) as compared to the amino acid sequence set forth in the SEQ ID NOs and retaining the binding affinity to TGFβ.

3. A humanized anti-TGFβ antibody, comprising the CDR1, the CDR2, and the CDR3 of the anti-TGFβ antibody according to claim 1, preferably comprising an FR region from Germline IGHV3-23*01 of human IgG, preferably further comprising an Fc constant region, wherein the constant region comprises a sequence set forth in SEQ ID NO: 18 or the human IgG1 heavy chain Fc constant region (GenBank No. AK303185.1), wherein more preferably, the humanized anti-TGFβ antibody comprises or consists of a humanized sequence set forth in SEQ ID NO: 20; further preferably, serine Ser at position 13 of a CDR2 of the humanized sequence set forth in SEQ ID NO: 20 is mutated to alanine Ala; most preferably, the humanized anti-TGFβ antibody comprises or consists of a humanized sequence selected from SEQ ID NOs: 21-23.

4. A bivalent or multivalent anti-TGFβ antibody, comprising 2 or more of the anti-TGFβ antibody according to claim 1, wherein preferably, the bivalent anti-TGFβ antibody has a sequence selected from the group consisting of: (1) the sequences set forth in SEQ ID NOs: 22, 19, 18, 19, and 22 connected end-to-end in order from the N-terminus to the C-terminus; (2) the sequences set forth in SEQ ID NOs: 4, 19, 18, 19, and 4 connected end-to-end in order from the N-terminus to the C-terminus; (3) the sequences set forth in SEQ ID NOs: 23, 19, 18, 19, and 23 connected end-to-end in order from the N-terminus to the C-terminus; (4) the sequences set forth in SEQ ID NOs: 21, 19, 18, 19, and 21 connected end-to-end in order from the N-terminus to the C-terminus; (5) the sequences set forth in SEQ ID NOs: 7, 19, 18, 19, and 7 connected end-to-end in order from the N-terminus to the C-terminus; and (6) the sequences set forth in SEQ ID NOs: 16, 19, 18, 19, and 16 connected end-to-end in order from the N-terminus to the C-terminus.

5. A multispecific antibody, comprising the anti-TGFβ antibody according to claim 1 and a second antibody against a second antigen selected from an immune cell surface antigen, a tumor antigen, a virus, a bacterium, an endotoxin, a cytokine, or a combination thereof, more preferably selected from: PD-L1, PD-1, VEGFA, IL-10, IL-10R, BCMA, VEGF, TGF-β, CTLA-4, LAG-3, TIGIT, CEA, CD38, SLAMF7, B7-H3, Her2, EpCAM, CD19, CD20, CD30, CD33, CD47, CD52, CD133, EGFR, GD2, GD3, GM2, RANKL, CD3, and/or CD16a, wherein preferably, the second antigen is VEGF; more preferably, the second antibody is selected from an anti-VEGF antibody comprising (i) an HCDR1 set forth in SEQ ID NO: 82, an HCDR2 set forth in SEQ ID NO: 83, an HCDR3 set forth in SEQ ID NO: 84, an LCDR1 set forth in SEQ ID NO: 85, an LCDR2 set forth in SEQ ID NO: 86, and an LCDR3 set forth in SEQ ID NO: 87, or (ii) an HCDR1 set forth in SEQ ID NO: 88, an HCDR2 set forth in SEQ ID NO: 89, an HCDR3 set forth in SEQ ID NO: 90, an LCDR1 set forth in SEQ ID NO: 91, an LCDR2 set forth in SEQ ID NO: 92, and an LCDR3 set forth in SEQ ID NO: 93; more preferably, the anti-VEGF antibody comprises (a) a heavy chain variable region set forth in SEQ ID NO: 24 or a sequence having at least 80%, 85%, 90%, 91%, 92%, 93%, 94%, 95%, 96%, 97%, 98%, or 99% homology to the heavy chain variable region set forth in SEQ ID NO: 24, and a light chain variable region set forth in SEQ ID NO: 27 or a sequence having at least 80%, 85%, 90%, 91%, 92%, 93%, 94%, 95%, 96%, 97%, 98%, or 99% homology to the light chain variable region set forth in SEQ ID NO: 27, or (b) a heavy chain variable region set forth in SEQ ID NO: 29 or a sequence having at least 80%, 85%, 90%, 91%, 92%, 93%, 94%, 95%, 96%, 97%, 98%, or 99% homology to the heavy chain variable region set forth in SEQ ID NO: 29, and a light chain variable region set forth in SEQ ID NO: 30 or a sequence having at least 80%, 85%, 90%, 91%, 92%, 93%, 94%, 95%, 96%, 97%, 98%, or 99% homology to the light chain variable region set forth in SEQ ID NO: 30; most preferably, the anti-VEGF antibody further comprises a CH1 set forth in SEQ ID NO: 25, a hinge region set forth in SEQ ID NO: 26, an Fc constant region set forth in SEQ ID NO: 18, and a light chain constant region set forth in SEQ ID NO: 28.

6. A polynucleotide, encoding the anti-TGFβ antibody according claim 1.

7. A vector, comprising the polynucleotide according to claim 6.

8. A host cell, comprising the vector according to claim 7.

9. A conjugate, comprising the anti-TGFβ antibody according to claim 1, and a conjugated moiety, wherein the conjugated moiety is a purification tag (e.g., His tag), a detectable label, a drug, a toxin, a cytokine, an enzyme or a combination thereof; preferably, the conjugated moiety is a radioisotope, a fluorescent substance, a chemiluminescent substance, a colored substance, a chemotherapeutic agent, a biotoxin, polyethylene glycol or an enzyme.

10. (canceled)

11. A pharmaceutical composition, comprising the anti-TGFβ antibody according to claim 1 a pharmaceutically acceptable carrier and/or excipient; preferably, the pharmaceutical composition is in a form suitable for administration through subcutaneous injection, intradermal injection, intravenous injection, intramuscular injection or intralesional injection.

12. A method for treating and/or preventing a tumor (e.g., treating a solid tumor), diabetes, systemic scleroderma, nephropathy, idiopathic pulmonary fibrosis, and/or multiple fibrosis in a subject in need thereof, comprising administering to the subject the anti-TGFβ antibody according to claim 1.


SEQUENCE LISTING (93 sequences)

SEQ ID NO: 1 - protein, 116 aa, Artificial sequence
  Synthetic
QVQLVESGGGLVQPGGSLRLSCAASGITFSHYDMAWFRQAPEKQRELVALITSGGKTAYADSVKGRFAISRDNAKNTVYLQMNSLKAGDTAVYYCNAHWVLTSQYWGQGTQVTVSS

SEQ ID NO: 2 - protein, 120 aa, Artificial sequence
  Synthetic
QLQLVESGGGLVQPGGSLRLSCTASGSTFEIYPIYAMAWFRQTPGKQRELVALTTDVKTNYADSVKGRFTISRDNAKNTVYLQMNMLQPEDTAVYVCNVGVVRGGLQNYWGQGTQVTVSS

SEQ ID NO: 3 - protein, 118 aa, Artificial sequence
  Synthetic
EVQVVESGGGLVQPGGSLRLSCAASASVFSWSVMGWFRQAPGKQRELVAISSGDRETYADSVKGRFTISRDNAKNTVYLQMDTLKPEDTAVYFCNAYVPVGESRGDYWGQGTQVTVSS

SEQ ID NO: 4 - protein, 118 aa, Artificial sequence
  Synthetic
EVQVVESGGGLVQPGGSLTLSCVASGFAFSFTPMRWVRQAPGKGLEWVSSISIRGDTTDYADSVKGRFTISRDNAKNTLYLQMNSLKPEDTAVYYCSRGSSISTSTERTQGTQVTVSS

SEQ ID NO: 5 - protein, 114 aa, Artificial sequence
  Synthetic
QVQLVESGGGLVQPGGSLRLSCAASGSIFGVFYMGWFRQAPGKQREMVAISTDDRTSYVDSVKGRFTISRDNAKNTLALQMNSLKPEDTAVYFCNLNQLGTDYWGPGTQVTVSS

SEQ ID NO: 6 - protein, 123 aa, Artificial sequence
  Synthetic
EVQLVESGGGLVQAGGSLRLSCAASGRSFSSLAVGWFRQPPGKKREIVAAVNWSGTSTYYPDSVKGRFSISRDNTKNTVYLQMNSLKPEDTAVYYCAADPDLRWLKLWGYEYWGQGTQVTVSS

SEQ ID NO: 7 - protein, 116 aa, Artificial sequence
  Synthetic
QVQLVESGGGLVQPGGSLRLSCAASGISFSYYDMGWFRQAPGKQRELVAIVTSGGRTNYADSVKGRFTISRETAKSTMYLQMNSLKSEDTGVYYCNAHWIVTTDYWGQGTQVTVAS

SEQ ID NO: 8 - protein, 118 aa, Artificial sequence
  Synthetic
QLQLVESGGGLVQPGGSLRLSCAGSGIAFSRVTMAWSRQAPGKQRELVALITDGNRSAYVDSVKGRFTISRDNTKKTMSLQMNDLKPEDTAVYYCVATDWVPTRRDYWGQGTQVTVSS

SEQ ID NO: 9 - protein, 114 aa, Artificial sequence
  Synthetic
QVQLVESGGGLVQPGGSLTLSCTASGSMFSFYTMGWYRQATGKQRELVAGISLGGETTYAPSVKDRFTISRDNAKNTVYLQMNSLKPEDTALYNCNAYWGEDYWGQGTQVTVSS

SEQ ID NO: 10 - protein, 126 aa, Artificial sequence
  Synthetic
EVQLVESGGGEVRPGGSLRLSCVVSGFTKDYGAIGWFRQAPGQEREGLGCIARKDGDTWSADSVKGRFTISRDNVRNTVYLQMNALKPEDRGVYYCAGDPSALYGCSDRSSEYQYWGQGTQVTVSS

SEQ ID NO: 11 - protein, 118 aa, Artificial sequence
  Synthetic
EVQLVESGGGLVQPGGSLRLSCAASGFTFSYYVASWYRQAPGKERELVATTFPPDGSPHYADSVKGRFAISRDNAKNTVFLQMDSLKSEDTAVYYCNARRVEGHGIYWGQGTQVTVSS

SEQ ID NO: 12 - protein, 114 aa, Artificial sequence
  Synthetic
QLQLVESGGGLVQPGESLRLSCAASGIIFSASTMAWFRQAPGKQRELVALITDGDRTNYADSVKGRFTISRDNAENTVYLQMTELKFEDTAVYYCNAFWLKAYWGQGTQVTVSS

SEQ ID NO: 13 - protein, 118 aa, Artificial sequence
  Synthetic
EVQVVESGGAAVQPGESLRLSCAASGSIFNTATMGWFRQAPGKQREMVAIITDGGRTNYADSVKGRFTISRDATKNTVYLQMNSLNPGDTAVYYCNALTIVPRSSIYWGQGTQVTVSS

SEQ ID NO: 14 - protein, 119 aa, Artificial sequence
  Synthetic
QLQLVESGGGLVQPGGSLTLSCLASGFTFSHYPMSWVRQAPGKGLEWVSVIGSDGAIKHYSDSVKGRFAISRDNAKNTLYLQMNSLKPEDTAVYYCAEGDSRYVRNRARGQGTQVTVSS

SEQ ID NO: 15 - protein, 119 aa, Artificial sequence
  Synthetic
QLQLVESGGGLVQPGGSLTLSCAASGFTFSHYPMSWVRQAPGKGLEWVSVIGSDGGTKIYSDSVKGRFAIARDNAKNTLYLQMNSLKPEDTAVYYCAEGDSRYVRGRTRGQGTQVTVSS

SEQ ID NO: 16 - protein, 114 aa, Artificial sequence
  Synthetic
QVQLVESGGGLVQPGESLRLSCTASGSIFGVFYMGWFRQAPGKQRDMVAISTDDRTSYADSVKGRFTISRDNAKNTLALQMNSLKPEDTAVYFCNLNQLGTDYWGPGTQVTVSS

SEQ ID NO: 17 - protein, 119 aa, Artificial sequence
  Synthetic
QLQLVESGGGLVQPGGSLTLSCAASGFTFSLYPMSWVRQAPGKGLEWVSVIGSDGGTKHYSDSVKGRFAISRDNAKNTLYLQMNSLKPEDTAVYYCAEGDSRYVRGRTRGQGTQVTVSS

SEQ ID NO: 18 - protein, 225 aa, Artificial sequence
  Synthetic
DKTHTCPPCPAPPVAGPSVFLFPPKPKDTLMISRTPEVTCVVVDVSHEAPEVQFNWYVDGVEVHNAKTKPREEQFNSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 5 aa, Artificial sequence
  Synthetic
GGGGS

SEQ ID NO: 20 - protein, 116 aa, Artificial sequence
  Synthetic
EVQLLESGGGLVQPGGSLRLSCAASGISFSYYDMGWFRQAPGKQRELVSIVTSGGRTNYADSVKGRFTISRDNSKSTLYLQMNSLRAEDTGVYYCNAHWIVTTDYWGQGTLVTVSS

SEQ ID NO: 21 - protein, 116 aa, Artificial sequence
  Synthetic
EVQLLESGGGLVQPGGSLRLSCAASGISFSYYDMGWFRQAPGKQRELVSIVTSGGRTNYADAVKGRFTISRDNSKSTLYLQMNSLRAEDTGVYYCNAHWIVTTDYWGQGTLVTVSS

SEQ ID NO: 22 - protein, 116 aa, Artificial sequence
  Synthetic
EVQLLESGGGLVQPGGSLRLSCAASGISFSYYDMGWFRQAPGKQRELVSIVTSGGRTNYADAVKGRFTISRDNSKNTLYLQMNSLRAEDTGVYYCNAHWIVTTDYWGQGTLVTVSS

SEQ ID NO: 23 - protein, 116 aa, Artificial sequence
  Synthetic
EVQLLESGGGLVQPGGSLRLSCAASGISFSYYDMGWFRQAPGKQRELVSIVTSGGRTNYADAVKGRFTISRDNSKSTLYLQMNSLRAEDTAVYYCNAHWIVTTDYWGQGTLVTVSS

SEQ ID NO: 24 - protein, 120 aa, Artificial sequence
  Synthetic
EVQLVESGGGLVQPGGSLRLSCAASGFTISDYWIHWVRQAPGKGLEWVAGITPAGGYTYYADSVKGRFTISADTSKNTAYLQMNSLRAEDTAVYYCARFVFFLPYAMDYWGQGTLVTVSS

SEQ ID NO: 25 - protein, 103 aa, Artificial sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 26 - protein, 7 aa, Artificial sequence
  Synthetic
DKTHTCP

SEQ ID NO: 27 - protein, 107 aa, Artificial sequence
  Synthetic
DIQMTQSPSSLSASVGDRVTITCRASQDVSTAVAWYQQKPGKAPKLLIYSASFLYSGVPSRFSGSGSGTDFTLTISSLQPEDFATYYCQQGYGNPPTFGQGTKVEIK

SEQ ID NO: 28 - protein, 107 aa, Artificial sequence [sequence omitted]

SEQ ID NO: 29 - protein, 123 aa, Artificial sequence [sequence omitted]

SEQ ID NO: 30 - protein, 107 aa, Artificial sequence [sequence omitted]

SEQ ID NO: 31 - protein, 10 aa, Artificial sequence
  Synthetic
GITFSHYDMA

SEQ ID NO: 32 - protein, 15 aa, Artificial sequence
  Synthetic
ITSGGKTAYADSVKG

SEQ ID NO: 33 - protein, 10 aa, Artificial sequence
  Synthetic
NAHWVLTSQY

SEQ ID NO: 34 - protein, 13 aa, Artificial sequence
  Synthetic
GSTFEIYPIYAMA

SEQ ID NO: 35 - protein, 15 aa, Artificial sequence
  Synthetic
LTTDVKTNYADSVKG

SEQ ID NO: 36 - protein, 12 aa, Artificial sequence
  Synthetic
NVGVVRGGLQNY

SEQ ID NO: 37 - protein, 10 aa, Artificial sequence
  Synthetic
ASVFSWSVMG

SEQ ID NO: 38 - protein, 15 aa, Artificial sequence
  Synthetic
ISSGDRETYADSVKG

SEQ ID NO: 39 - protein, 13 aa, Artificial sequence
  Synthetic
NAYVPVGESRGDY

SEQ ID NO: 40 - protein, 10 aa, Artificial sequence
  Synthetic
GFAFSFTPMR

SEQ ID NO: 41 - protein, 17 aa, Artificial sequence
  Synthetic
SISIRGDTTDYADSVKG

SEQ ID NO: 42 - protein, 11 aa, Artificial sequence
  Synthetic
SRGSSISTSTE

SEQ ID NO: 43 - protein, 10 aa, Artificial sequence
  Synthetic
GSIFGVFYMG

SEQ ID NO: 44 - protein, 15 aa, Artificial sequence
  Synthetic
ISTDDRTSYVDSVKG

SEQ ID NO: 45 - protein, 9 aa, Artificial sequence
  Synthetic
NLNQLGTDY

SEQ ID NO: 46 - protein, 10 aa, Artificial sequence
  Synthetic
GRSFSSLAVG

SEQ ID NO: 47 - protein, 17 aa, Artificial sequence
  Synthetic
AVNWSGTSTYYPDSVKG

SEQ ID NO: 48 - protein, 16 aa, Artificial sequence
  Synthetic
AADPDLRWLKLWGYEY

SEQ ID NO: 49 - protein, 10 aa, Artificial sequence
  Synthetic
GISFSYYDMG

SEQ ID NO: 50 - protein, 16 aa, Artificial sequence
  Synthetic
IVTSGGRTNYADSVKG

SEQ ID NO: 51 - protein, 10 aa, Artificial sequence
  Synthetic
NAHWIVTTDY

SEQ ID NO: 52 - protein, 10 aa, Artificial sequence
  Synthetic
GIAFSRVTMA

SEQ ID NO: 53 - protein, 15 aa, Artificial sequence
  Synthetic
ITDGNRSAYVDSVKG

SEQ ID NO: 54 - protein, 12 aa, Artificial sequence
  Synthetic
VATDWVPTRRDY

SEQ ID NO: 55 - protein, 8 aa, Artificial sequence
  Synthetic
GSMFSFYT

SEQ ID NO: 56 - protein, 15 aa, Artificial sequence
  Synthetic
ISLGGETTYAPSVKD

SEQ ID NO: 57 - protein, 8 aa, Artificial sequence
  Synthetic
NAYWGEDY

SEQ ID NO: 58 - protein, 10 aa, Artificial sequence
  Synthetic
GFTKDYGAIG

SEQ ID NO: 59 - protein, 16 aa, Artificial sequence
  Synthetic
IARKDGDTWSADSVKG

SEQ ID NO: 60 - protein, 19 aa, Artificial sequence
  Synthetic
AGDPSALYGCSDRSSEYQY

SEQ ID NO: 61 - protein, 10 aa, Artificial sequence
  Synthetic
GFTFSYYVAS

SEQ ID NO: 62 - protein, 17 aa, Artificial sequence
  Synthetic
TTFPPDGSPHYADSVKG

SEQ ID NO: 63 - protein, 11 aa, Artificial sequence
  Synthetic
NARRVEGHGIY

SEQ ID NO: 64 - protein, 10 aa, Artificial sequence
  Synthetic
GIIFSASTMA

SEQ ID NO: 65 - protein, 16 aa, Artificial sequence
  Synthetic
LITDGDRTNYADSVKG

SEQ ID NO: 66 - protein, 8 aa, Artificial sequence
  Synthetic
NAFWLKAY

SEQ ID NO: 67 - protein, 10 aa, Artificial sequence
  Synthetic
GSIFNTATMG

SEQ ID NO: 68 - protein, 16 aa, Artificial sequence
  Synthetic
IITDGGRTNYADSVKG

SEQ ID NO: 69 - protein, 12 aa, Artificial sequence
  Synthetic
NALTIVPRSSIY

SEQ ID NO: 70 - protein, 10 aa, Artificial sequence
  Synthetic
GFTFSHYPMS

SEQ ID NO: 71 - protein, 16 aa, Artificial sequence
  Synthetic
IGSDGAIKHYSDSVKG

SEQ ID NO: 72 - protein, 12 aa, Artificial sequence
  Synthetic
AEGDSRYVRNRA

SEQ ID NO: 73 - protein, 10 aa, Artificial sequence
  Synthetic
GFTFSHYPMS

SEQ ID NO: 74 - protein, 16 aa, Artificial sequence
  Synthetic
IGSDGGTKIYSDSVKG

SEQ ID NO: 75 - protein, 12 aa, Artificial sequence
  Synthetic
AEGDSRYVRGRT

SEQ ID NO: 76 - protein, 10 aa, Artificial sequence
  Synthetic
GSIFGVFYMG

SEQ ID NO: 77 - protein, 15 aa, Artificial sequence
  Synthetic
ISTDDRTSYADSVKG

SEQ ID NO: 78 - protein, 9 aa, Artificial sequence
  Synthetic
NLNQLGTDY

SEQ ID NO: 79 - protein, 10 aa, Artificial sequence
  Synthetic
GFTFSLYPMS

SEQ ID NO: 80 - protein, 16 aa, Artificial sequence
  Synthetic
IGSDGGTKHYSDSVKG

SEQ ID NO: 81 - protein, 12 aa, Artificial sequence
  Synthetic
AEGDSRYVRGRT

SEQ ID NO: 82 - protein, 5 aa, Artificial sequence
  Synthetic
DYWIH

SEQ ID NO: 83 - protein, 17 aa, Artificial sequence
  Synthetic
GITPAGGYTYYADSVKG

SEQ ID NO: 84 - protein, 11 aa, Artificial sequence
  Synthetic
FVFFLPYAMDY

SEQ ID NO: 85 - protein, 11 aa, Artificial sequence
  Synthetic
RASQDVSTAVA

SEQ ID NO: 86 - protein, 7 aa, Artificial sequence
  Synthetic
SASFLYS

SEQ ID NO: 87 - protein, 9 aa, Artificial sequence
  Synthetic
QQGYGNPPT

SEQ ID NO: 88 - protein, 10 aa, Artificial sequence
  Synthetic
GYTFTNYGMN

SEQ ID NO: 89 - protein, 17 aa, Artificial sequence
  Synthetic
WINTYTGEPTYAADFKR

SEQ ID NO: 90 - protein, 14 aa, Artificial sequence
  Synthetic
YPHYYGSSHWYFDV

SEQ ID NO: 91 - protein, 11 aa, Artificial sequence
  Synthetic
SASQDISNYLN

SEQ ID NO: 92 - protein, 7 aa, Artificial sequence
  Synthetic
FTSSLHS

SEQ ID NO: 93 - protein, 9 aa, Artificial sequence
  Synthetic
QQYSTVPWT